(19) United States
(12) Patent Application Publication
(10) Pub. No.: US 2010/0227863 A1
(43) Pub. Date: Sep. 9, 2010

(54) SUBSTITUTED 3-(4-HYDROXYPHENYL)-INDOLIN-2-ONE COMPOUNDS

(75) Inventors: Mette Knak Christensen, Holte (DK); Fredrik Bjoerkling, Helsingborg (SE)

Correspondence Address: DARBY & DARBY P.C., P.O. BOX 770, Church Street Station, New York, NY, 10008-0770, US

(73) Assignee: TOPOTARGET A/S, Copenhagen (DK)

(21) Appl. No.: 12/597,208

(22) PCT Filed: Apr. 28, 2008
(86) PCT No.: PCT/EP08/54990

§ 371 Date: Apr. 16, 2010

Related U.S. Application Data
(60) Provisional application No. 60/913,625, filed on Apr. 24, 2007.

(51) Int. Cl. A61K 31/5377; C07D 209/34; A61K 31/4015; C07D 401/04; A61K 31/4439; C07D 413/04; A61P 35/00

(52) U.S. Cl. 514/235.2; 548/486; 514/418; 546/277.7; 514/339; 544/144

(57) ABSTRACT

The present application discloses substituted 3-(4-hydroxyphenyl)-indolin-2-one compounds (oxindole compounds) of the formula

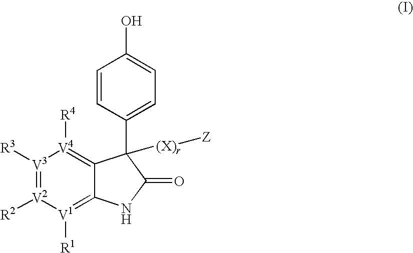

and the use of such compounds for the preparation of a medicament for the treatment of cancer in a mammal, in particular in humans.

### FIELD OF THE INVENTION

The present invention relates to novel substituted 3-(4-hydroxyphenyl)-indolin-2-one compounds (oxindole compounds), and the use of such compounds for the preparation of a medicament for the treatment of cancer in a mammal.

### BACKGROUND OF THE INVENTION

U.S. Pat. No. 1,624,675 describes O—O-diacyl derivatives of diphenolisatine and that these compounds possess laxative properties.

US 2004/0242563 A 1 discloses substituted diphenyl indanone, indane and indole compounds and analogues thereof useful for the treatment or prevention of diseases characterized by abnormal cell proliferation.

Magnus et al. (Magnus P and Turnbull R (2006) Organic Letters 8(16): 3497-3499) describe the synthesis of the following oxindole:

| Structure | CAS No. |
|---|---|
| 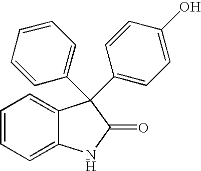 | 685890-84-0P |

Felding et al. (WO 2005/097107) describe a number of oxindoles as anti-cancer agents, e.g. the following oxindoles:

| Structure | CAS No. |
|---|---|
| 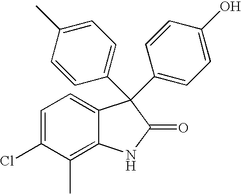 | 867154-97-0P |
| 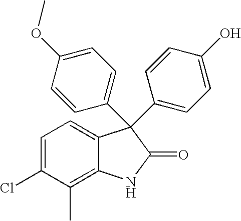 | 867154-98-1P |
| 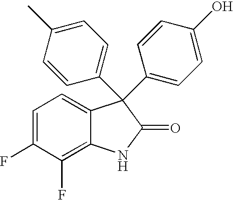 | 867154-99-2P |
| 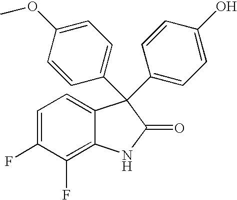 | 867155-00-8P |
| 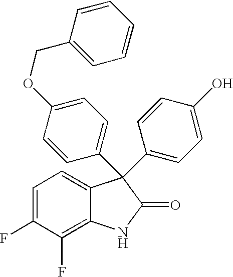 | 867155-02-0P |
| 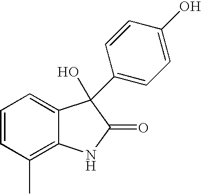 | 867155-03-1P |
| 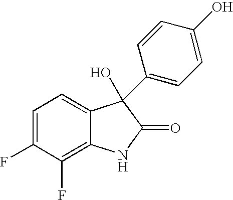 | 867155-04-2P |

Halperin et al. (WO 2005/080335) describe a number of oxindoles as potential anti-cancer agents, e.g. the following oxindoles;

| Structure | CAS No. |
|---|---|
| 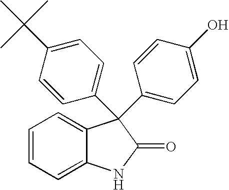 | 685890-93-1P |
| 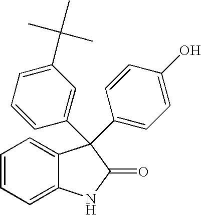 | 863779-78-6P |
| 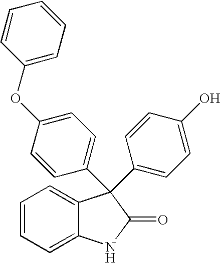 | 863779-79-7P |
| 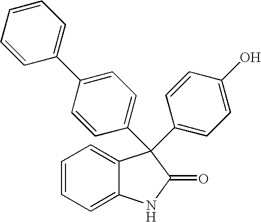 | 863779-80-0P |
| 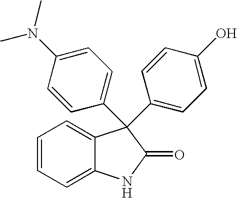 | 863779-81-1P |

Baskakova et al. (SU 90-4875262) describe the following oxindole for the manufacture of optical articles.

| Structure | CAS No. |
|---|---|
| 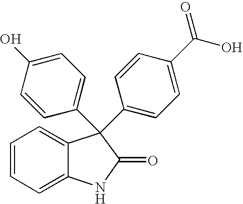 | 189168-16-9D |

Kawada et al. (JP 94-114510) describe the following oxindole as a resist agent:

| Structure | CAS No. |
|---|---|
| 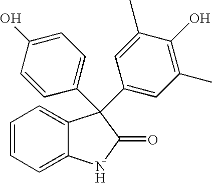 | 174572-24-8 |

Hosta Pujol et al. (DE 2521966) describe the synthesis of the following oxindole as a potential laxative:

| Structure | CAS No. |
|---|---|
| 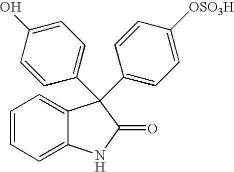 | 56632-45-2P |

Esteve Subirana et al. (DE 2451592) describe the following oxindole as a laxative agent:

| Structure | CAS No. |
|---|---|
| 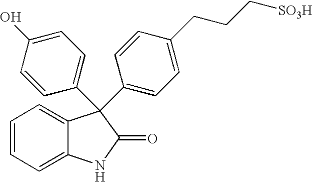 | 57352-72-4P |

Kornowski (Kornowski H (1963) Bulletin de la Societe Chimique de France 10: 2035-2036) describes the synthesis of the following oxindole:

| Structure | CAS No. |
|---|---|
| 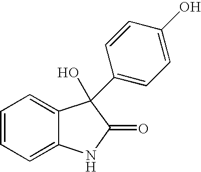 | 92163-66-1P |

Aktiebolaget “Ferrosan” ((1957) British patent application no. GB 1955-34509) describes the following oxindole as a laxative:

| Structure | CAS No. |
|---|---|
| 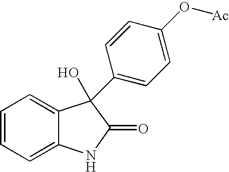 | 861221-88-7 |

Geigy J R ((1955) British patent application no. GB 1952-23426) describes the synthesis of the following oxindoles:

| Structure | CAS No. |
|---|---|
| 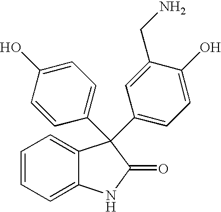 | 855420-69-8 |
| 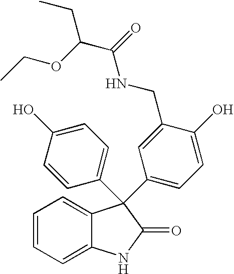 | 854425-57-3P |
| 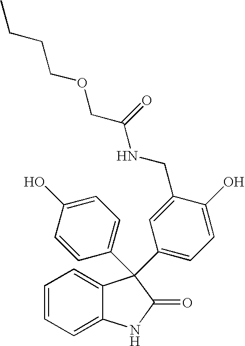 | 855929-75-8P |
| 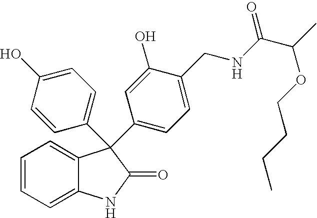 | 857235-30-4P |
| 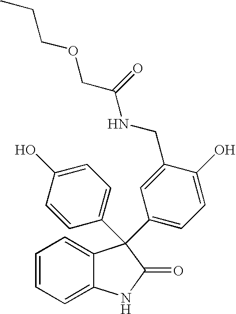 | 857945-23-4P |
| 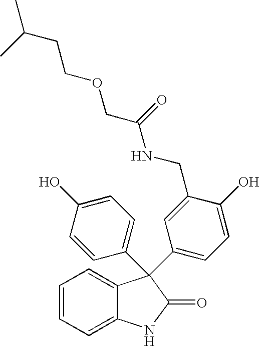 | 857945-24-5P |
| 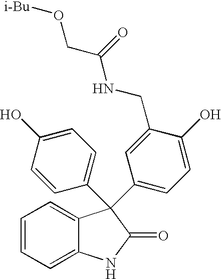 | 857945-25-6P |
| 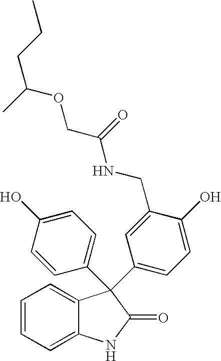 | 859057-53-7P |
| 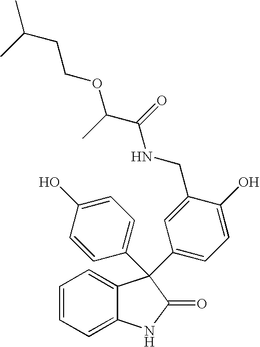 | 859301-30-7P |
| 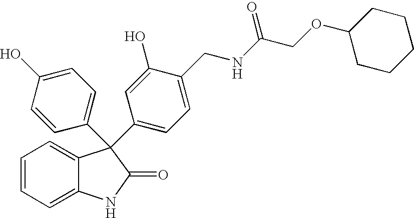 | 861055-20-1P |
| 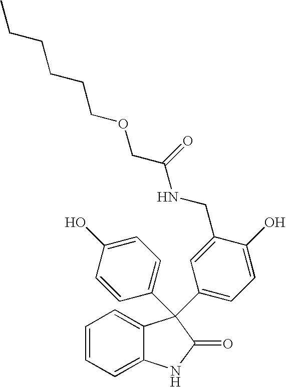 | 861058-00-6P |

Luk et al. (WO 2006/136606) describe oxindoles as potential anticancer agents:

Structure

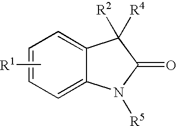

Notably, these compounds comprise only one R¹ substituent. Also neither R² nor R⁴ are para-hydroxyphenyl.

Although, Felding et al. and Halperin et al. describe various ox-indole-2-one-type compounds as anti-cancer agents, there is still a need for novel ox-indol-2-one-type compounds as anti-cancer agents which provide useful alternatives upon selection of drug candidates.

### BRIEF DESCRIPTION OF THE INVENTION

The present inventors have now found that a new class of compounds represents an excellent alternative to existing ox-indol-2-one-type compounds as anti-cancer agents, and that the new compounds have comparative or even improved potency compared to the known compounds.

Hence, the present invention provides compounds of the general formulae (I) and (Ia), cf. claims 1, 24 and 25.

The present invention further provides a pharmaceutical composition, cf. claim 26, the utilization of compounds of the general formulae (I) and (Ia) in medicine, cf. claims 28, 29 and 31.

### DETAILED DESCRIPTION OF THE INVENTION

### The Compounds of the General Formula (I)

The present invention i.a. relates to particular prodrug compounds which are useful for the treatment of cancer in a mammal.

The useful prodrug compounds have the general formula (I), namely

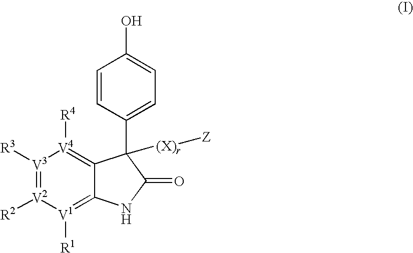

wherein
r is 0 or 1;
X is selected from —CH₂—, —O—, —S—, —S(O)—, —S(O)₂— and —NR⁵—, wherein R⁵ is selected from hydrogen and optionally substituted C₁₋₆-alkyl;
Z is selected from optionally substituted C₁₋₁₂-alkyl, optionally substituted C₃₋₁₂-cycloalkyl, optionally substituted C₂₋₁₂-alkenyl, optionally substituted C₃₋₁₂-cycloalkenyl, optionally substituted C₂₋₁₂-alkynyl, optionally substituted heterocyclyl, optionally substituted aryl and optionally substituted heteroaryl;
with the proviso that Z is not para-mono-substituted phenyl when r is 0, in particular not mono-substituted phenyl;
V¹, V², V³, and V⁴ independently are selected from a carbon atom, a non-quaternary nitrogen atom, an oxygen atom, and a sulphur atom, and where V⁴ further may be selected from a bond, so that -V¹-V²-V³-V⁴- together with the atoms to which V¹ and V⁴ are attached form an aromatic or heteroaromatic ring;
R¹, R², R³, and R⁴, when attached to a carbon atom, independently are selected from hydrogen, optionally substituted C₁₋₁₂-alkyl, optionally substituted C₃₋₁₂-cycloalkyl, optionally substituted C₂₋₁₂-alkenyl, optionally substituted C₃₋₁₂-cycloalkenyl, hydroxy, optionally substituted C₁₋₁₂-alkoxy, optionally substituted C₂₋₁₂-alkenyloxy, carboxy, optionally substituted C₁₋₁₂-alkoxycarbonyl, optionally substituted C₁₋₁₂-alkylcarbonyl, optionally substituted C₁₋₁₂-alkylcarbonyloxy, formyl, amino, mono- and di(C₁₋₁₂-alkyl)amino, carbamoyl, mono- and di(C₁₋₁₂-alkyl)aminocarbonyl, C₁₋₁₂-alkylcarbonylamino, C₁₋₁₂-alkylsulphonylamino, cyano, carbamido, mono- and di(C₁₋₁₂-alkyl)amino-carbonylamino, C₁₋₁₂-alkanoyloxy, C₁₋₁₂-alkylsulphonyl, C₁₋₁₂-alkylsulphinyl, aminosulphonyl, mono- and di(C₁₋₁₂-alkyl)aminosulphonyl, nitro, optionally substituted C₁₋₁₂-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₁₂-alkyl as an amino substituent is optionally substituted with hydroxy, C₁₋₁₂-alkoxy, amino, mono- and di(C₁₋₁₂-alkyl)amino, carboxy, C₁₋₁₂-alkylcarbonylamino, C₁₋₁₂-alkylaminocarbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₁₂-alkyl, hydroxy, oxide, optionally substituted C₁₋₁₂-alkoxy, optionally substituted C₁₋₁₂-alkoxycarbonyl, optionally substituted C₁₋₁₂-alkylcarbonyl, formyl, mono- and di(C₁₋₁₂-alkyl)aminocarbonyl, amino, C₁₋₁₂-alkylcarbonylamino, mono- and di(C₁₋₁₂-alkyl)amino, C₁₋₁₂-alkylsulphonyl, C₁₋₁₂-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₁₂-alkyl as an amino substituent is optionally substituted with hydroxy, C₁₋₁₂-alkoxy, amino, mono- and di(C₁₋₁₂-alkyl)amino, carboxy, C₁₋₁₂-alkylcarbonylamino, C₁₋₁₂-alkylaminocarbonyl, or halogen(s), and wherein any aryl, heterocyclyl and heteroaryl may be optionally substituted;
or R¹ and R² together with the carbon atoms to which they are attached form a ring;
with the proviso that at least one of the substituents R¹, R², R³, and R⁴ is not hydrogen;
and pharmaceutically acceptable salts and prodrugs thereof.

### DEFINITIONS

In the present context, the terms “C₁₋₁₂-alkyl” and “C₁₋₆-alkyl” are intended to mean a linear, cyclic or branched hydrocarbon group having 1 to 12 carbon atoms and 1 to 6 carbon atoms, respectively, such as methyl, ethyl, propyl, iso-propyl, pentyl, cyclopentyl, hexyl, cyclohexyl. The term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Although the term “C₃₋₁₂-cycloalkyl” is encompassed by the term “C₁₋₁₂-alkyl”, it refers specifically to the mono- and bicyclic counterparts, including alkyl groups having exo-cyclic atoms, e.g. cyclohexyl-methyl.

Similarly, the terms “C₂₋₁₂-alkenyl” and “C₂₋₆-alkenyl” are intended to cover linear, cyclic or branched hydrocarbon groups having 2 to 12 carbon atoms and 2 to 6 carbon atoms, respectively, and comprising (at least) one unsaturated bond. Examples of alkenyl groups are vinyl, allyl, butenyl, pentenyl, hexenyl, heptenyl, octenyl, heptadecaenyl. Preferred examples of alkenyl are vinyl, allyl, butenyl, especially allyl.

Although the term “C₃₋₁₂-cycloalkenyl” is encompassed by the term “C₂₋₁₂-alkenyl”, it refers specifically to the mono- and bicyclic counterparts, including alkenyl groups having exo-cyclic atoms, e.g. cyclohexenyl-methyl.

In the present context, i.e. in connection with the terms “alkyl”, “cycloalkyl”, “alkylidene”, “alkoxy”, “alkenyl”, “cycloalkenyl” and the like, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁₋₆-alkyl)amino, —N(C₁₋₄-alkyl)₃⁺, carbamoyl, mono- and di(C₁₋₆-alkyl)aminocarbonyl, C₁₋₆-alkyl-carbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl.

In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The term “halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydro-pyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “benzylidene”, “heteroaryl”, “heterocyclyl” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), oxide (only relevant as the N-oxide), carboxy, C₁₋₆-alkoxycarbonyl, C₁₋₆-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyl-oxy, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, nitro, sulphanyl, amino, amino-sulphonyl, mono- and di(C₁₋₆-alkyl)amino-sulphonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Typically, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-suphonyl, C₁₋₆-alkyl-sulphinyl, C₁₋₆-alkylsulphonyloxy, sulphanyl, amino, amino-sulphonyl, mono- and di(C₁₋₆-alkyl)amino-sulphonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The term “prodrug” used herein is intended to mean a compound which—upon exposure to physiological conditions—will liberate a derivative said compound which then will be able to exhibit the desired biological action.

The term “pharmaceutically acceptable salts” is intended to include acid addition salts and basic salts. Illustrative examples of acid addition salts are pharmaceutically acceptable salts formed with non-toxic acids. Exemplary of such organic salts are those with maleic, fumaric, benzoic, ascorbic, succinic, oxalic, bis-methylenesalicylic, methanesulphonic, ethanedisulphonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulphonic, and theophylline acetic acids, as well as the 8-halotheophyllines, for example 8-bromotheophylline. Exemplary of such inorganic salts are those with hydrochloric, hydrobromic, sulphuric, sulphamic, phosphoric, and nitric acids. Examples of basic salts are salts where the (remaining) counter ion is selected from alkali metals, such as sodium and potassium, alkaline earth metals, such as calcium, and ammonium ions (⁺N(R)₃R′, where R and R′ independently designates optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the compounds as well as any intermediates or starting materials may also be present in hydrate form.

Moreover, it should be understood that the compounds may be present as racemic mixtures or the individual stereoisomers such as enantiomers or diastereomers. The present invention encompasses each and every of such possible stereoisomers (e.g. enantiomers and diastereomers) as well as racemates and mixtures enriched with respect to one of the possible stereoisomers.

### EMBODIMENTS

It should be understood that relevant feature of the compounds of the formula (I) include that the group Z is not para-mono-substituted phenyl (in particular not mono-substituted) when r is 0, and at least one of the substituents R¹, R², R³, and R⁴ is not hydrogen. Preferably, at least two of the substituents R¹, R², R³, and R⁴ are not hydrogen;

It appears that the group Z (as defined hereinabove) plays an important role for the optimization of the biological activity of the compounds.

This being said, Z is in one interesting embodiment selected from optionally substituted C₁₋₁₂-alkyl, optionally substituted C₃₋₁₂-cycloalkyl, optionally substituted C₂₋₁₂-alkenyl, optionally substituted C₃₋₁₂-cycloalkenyl, optionally substituted C₂₋₁₂-alkynyl, and optionally substituted heterocyclyl.

In one variant hereof, Z is selected from C₁₋₁₂-alkyl, C₃₋₁₂-cycloalkyl, C₂₋₁₂-alkenyl, C₃₋₁₂-cycloalkenyl, and C₂₋₁₂-alkynyl.

In another variant hereof, Z is selected from optionally substituted C₃₋₁₂-cycloalkyl and optionally substituted heterocyclyl (e.g. piperidine and morpholine), in particular from C₃₋₁₂-cycloalkyl, heterocyclyl, and mono-substituted heterocyclyl.

In another interesting embodiment, Z is optionally substituted heteroaryl, in particular heteroaryl.

In a still further interesting embodiment, Z is aryl or, alternatively, Z is di- or tri-substituted aryl.

The orientation of the group Z is also in part defined by the presence (r=1) and type of the group X.

In one interesting embodiment, r is 1 and X is —CH₂—.

In another interesting embodiment, r is 0.

The atoms V¹, V², V³, and V⁴ define whether the ring is an aromatic or heteroaromatic ring. Besides an aromatic ring (a benzene ring), a plethora of aromatic rings are possible.

In one particularly interesting embodiment, however, each of V¹, V², V³, and V⁴ represents a carbon atom (a benzene ring), or V³ represents a nitrogen atom and each of V¹, V², and V⁴ represents a carbon atom (a pyridine ring). In the currently most interesting embodiments, each of V¹, V², V³, and V⁴ represents a carbon atom (i.e. the ring is a benzene ring).

The substituents R¹ and R² of the substituents R¹, R², R³, and R⁴ seem to play a particular role.

Preferably, R¹ is selected from halogen, C₁₋₆-alkyl, trifluoromethyl and C₁₋₆-alkoxy, when V¹ is a carbon atom.

Also preferably, R² is selected from halogen, optionally substituted C₁₋₆-alkyl, and optionally substituted C₁₋₆-alkoxy, when V² is a carbon atom.

Further, it is preferred that R³ is selected from hydrogen, optionally substituted C₁₋₆-alkoxy, halogen, cyano, optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulphonyl, when V³ is a carbon atom.

Even further, it is preferred that R⁴ is hydrogen, when V⁴ is a carbon atom.

This being said, it is preferred that at least two of the substituents R¹, R², R³, and R⁴ are not hydrogen.

In one variant hereof, R³ and R⁴ are both hydrogen.

In a further variant hereof, none of R¹ and R² are hydrogen. In a particular variant, R¹ and R² are both selected from halogen and methyl. In a specific variant hereof, R¹ and R² are both fluoro.

Alternatively, R¹ and R² together with the carbon atoms to which they are attached form a ring selected from aromatic rings, carbocyclic rings, heterocyclic rings and heteroaromatic rings, in particular aromatic rings, heterocyclic rings and heteroaromatic rings.

### The Compounds of General Formula (Ia)

It has been found that certain compounds wherein R³ and R⁴ are both hydrogen and wherein none of R¹ and R² are hydrogen represent a particularly interesting aspect of the present invention. Hence, the present invention also provides a compound of the general formula (Ia)

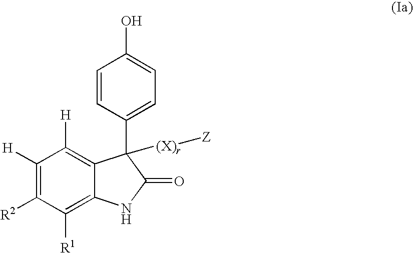

wherein Z, R¹ and R² are as defined herein, with the proviso that none of R¹ and R² are hydrogen.

### Currently Most Preferred Compounds

Presently very interesting compounds of the formulae (I) and (Ia) are those listed in the following:
3-ethynyl-6,7-difluoro-3-(4-hydroxyphenyl)indolin-2-one
3-benzyl-6,7-difluoro-3-(4-hydroxyphenyl)indoline-2-one
6,7-difluoro-3-(4-hydroxyphenyl)-3-methylindolin-2-one
3-cyclopentyl-6,7-difluoro-3-(4-hydroxyphenyl)indolin-2-one
3-(cyclohexylmethyl)-6,7-difluoro-3-(4-hydroxyphenyl)indolin-2-one
6,7-difluoro-3-(4-hydroxyphenyl)-3-(pyridin-4-yl)indolin-2-one
6,7-difluoro-3-(4-hydroxyphenyl)-3-isopropylindolin-2-one
6,7-difluoro-3-(4-hydroxyphenyl)-3-(thiophen-2-yl)indolin-2-one
3-butyl-6,7-difluoro-3-(4-hydroxyphenyl)indolin-2-one
3-cyclohexyl-6,7-difluoro-3-(4-hydroxyphenyl)indolin-2-one
6,7-difluoro-3-(4-hydroxyphenyl)-3-propylindolin-2-one
6,7-difluoro-3-(4-hydroxyphenyl)-3-pentylindolin-2-one
6,7-difluoro-3-(4-hydroxyphenyl)-3-pentylindolin-2-one
6,7-difluoro-3-(4-hydroxyphenyl)-3-(pyridin-3-yl)indolin-2-one
6,7-difluoro-3-(4-hydroxyphenyl)-3-(pyridin-4-yl N-oxide)indolin-2-one
3-(but-3-en-2-yl)-6,7-difluoro-3-(4-hydroxyphenyl)indolin-2-one
3-sec-butyl-6,7-difluoro-3-(4-hydroxyphenyl)indolin-2-one
3-cycloheptyl-6,7-difluoro-3-(4-hydroxyphenyl)indolin-2-one
3-(1-(benzyloxy)-1H-pyrazol-4-yl)-6,7-difluoro-3-(4-hydroxyphenyl)indolin-2-one
6,7-difluoro-3-(1-hydroxy-1H-pyrazol-4-yl)-3-(4-hydroxyphenyl)indolin-2-one
3-cyclohexyl-3-(4-hydroxyphenyl)-7-(trifluoromethyl)indolin-2-one
3-(3,4-difluorophenyl)-6,7-difluoro-3-(4-hydroxyphenyl)indolin-2-one
6,7-difluoro-3-(3-fluor-4-methylphenyl)-3-(4-hydroxyphenyl)indolin-2-one
6-chloro-3-cyclohexyl-3-(4-hydroxyphenyl)-7-methylindolin-2-one
3-cyclohexyl-3-(4-hydroxyphenyl)-6,7-dimethylindolin-2-one
3-(cyclopentylmethyl)-6,7-difluoro-3-(4-hydroxyphenyl)indolin-2-one
3-cyclohexyl-6,7-difluoro-3-(4-hydroxyphenyl)indolin-2-one (R-enantiomer)
3-cyclohexyl-6,7-difluoro-3-(4-hydroxyphenyl)indolin-2-one (S-enantiomer)
6,7-difluoro-3-(4-hydroxy-3-methylphenyl)-3-(4-hydroxyphenyl)indolin-2-one
6,7-difluoro-3-(4-hydroxy-2-methylphenyl)-3-(4-hydroxyphenyl)indolin-2-one
3-cyclooctyl-6,7-difluoro-3-(4-hydroxyphenyl)indolin-2-one
6,7-difluoro-3-(4-hydroxyphenyl)-3-(naphthalene-1-yl)indolin-2-one
3-cyclohexyl-7-fluoro-3-(4-hydroxyphenyl)-6-methylindolin-2-one
3-tert-butyl-6,7-difluoro-3-(4-hydroxyphenyl)indolin-2-one
6,7-difluoro-3-(4-hydroxyphenyl)-3-tert-pentylindolin-2-one
3-cyclopentyl-6-fluoro-3-(4-hydroxyphenyl)-7-methylindolin-2-one
3-cyclohexyl-6-fluoro-3-(4-hydroxyphenyl)-7-methylindolin-2-one
6-fluoro-3-(4-hydroxyphenyl)-7-methyl-3-pentylindolin-2-one
3-cycloheptyl-6-fluoro-3-(4-hydroxyphenyl)-7-methylindolin-2-one
3-cycloheptyl-3-(4-hydroxyphenyl)-7-(trifluoromethyl)indolin-2-one
3-cycloheptyl-3-(4-hydroxyphenyl)-6,7-dimethylindolin-2-one
6-chloro-3-cycloheptyl-3-(4-hydroxyphenyl)-7-methylindolin-2-one
3-cyclopentyl-3-(4-hydroxyphenyl)-6-methoxy-7-methylindolin-2-one
3-cyclohexyl-3-(4-hydroxyphenyl)-6-methoxy-7-methylindolin-2-one
3-(4-hydroxyphenyl)-3-(1H-imidazol-1-yl)-7-(trifluoromethyl)indolin-2-one
6,7-difluoro-3-(4-hydroxyphenyl)-3-(1H-imidazol-1-yl)indolin-2-one
6,7-difluoro-3-(4-hydroxyphenyl)-3-morpholinoindolin-2-one
3-(4-hydroxyphenyl)-3-(thiazol-2-yl)-7-(trifluoromethyl)indolin-2-one
7-chloro-3-cyclohexyl-3-(4-hydroxyphenyl)-6-methylindolin-2-one
7-chloro-3-cyclopentyl-3-(4-hydroxyphenyl)-6-methylindolin-2-one
7-chloro-3-cycloheptyl-3-(4-hydroxyphenyl)-6-methylindolin-2-one)
3-cyclohexyl-6-hydroxy-3-(4-hydroxyphenyl)-7-methylindolin-2-one
7-bromo-3-cyclopentyl-3-(4-hydroxyphenyl)-6-methylindolin-2-one
7-bromo-3-cyclohexyl-3-(4-hydroxyphenyl)-6-methylindolin-2-one
7-bromo-3-cycloheptyl-3-(4-hydroxyphenyl)-6-methylindolin-2-one
3-cyclopentyl-3-(4-hydroxyphenyl)-7-methylindolin-2-one
3-cyclohexyl-3-(4-hydroxyphenyl)-7-methylindolin-2-one
3-cycloheptyl-3-(4-hydroxyphenyl)-7-methylindolin-2-one
7-bromo-3-cyclopentyl-3-(4-hydroxyphenyl)indolin-2-one
7-bromo-3-cyclohexyl-3-(4-hydroxyphenyl)indolin-2-one
7-bromo-3-cycloheptyl-3-(4-hydroxyphenyl)indolin-2-one
3-cyclooctyl-3-(4-hydroxyphenyl)-7-(trifluoromethyl)indolin-2-one
7-chloro-3-cyclooctyl-3-(4-hydroxyphenyl)-6-methylindolin-2-one
3-cyclopentyl-3-(4-hydroxyphenyl)-5,7-dimethylindolin-2-one
3-cyclohexyl-3-(4-hydroxyphenyl)-5,7-dimethylindolin-2-one
3-cycloheptyl-3-(4-hydroxyphenyl)-5,7-dimethylindolin-2-one
3-cyclopentyl-3-(4-hydroxyphenyl)-5-methoxy-7-methylindolin-2-one
3-cyclohexyl-3-hydroxy-5-methoxy-7-methylindolin-2-one
3-cycloheptyl-3-(4-hydroxyphenyl)-5-methoxy-7-methylindolin-2-one
5-chloro-3-cyclopentyl-3-(4-hydroxyphenyl)-7-methylindolin-2-one
5-chloro-3-cyclohexyl-3-(4-hydroxyphenyl)-7-methylindolin-2-one
5-chloro-3-cycloheptyl-3-hydroxy-7-methylindolin-2-one
3-cyclopentyl-5-fluoro-3-(4-hydroxyphenyl)-7-methylindolin-2-one
3-cyclohexyl-5-fluoro-3-(4-hydroxyphenyl)-7-methylindolin-2-one
3-cycloheptyl-5-fluoro-3-(4-hydroxyphenyl)-7-methylindolin-2-one
3-cyclopentyl-6-fluoro-3-(4-hydroxyphenyl)-5,7-dimethylindolin-2-one
3-cyclohexyl-6-fluoro-3-(4-hydroxyphenyl)-5,7-dimethylindolin-2-one
3-cycloheptyl-6-fluoro-3-(4-hydroxyphenyl)-5,7-dimethylindolin-2-one
3-cyclopentyl-3-(4-hydroxyphenyl)-7-methyl-6-(trifluoromethyl)indolin-2-one
3-cyclohexyl-3-(4-hydroxyphenyl)-7-methyl-6-(trifluoromethyl)indolin-2-one
3-cycloheptyl-3-(4-hydroxyphenyl)-7-methyl-6-(trifluoromethyl)indolin-2-one
3-cyclopentyl-3-(4-hydroxyphenyl)-5-methoxy-6,7-dimethylindolin-2-one
3-cyclohexyl-3-(4-hydroxyphenyl)-5-methoxy-6,7-dimethylindolin-2-one
3-cycloheptyl-3-(4-hydroxyphenyl)-5-methoxy-6,7-dimethylindolin-2-one

### Preparation of Compounds

The compounds of the present invention can be prepared in a number of ways well known to those skilled in the art of organic synthesis. The compounds of the present invention can be synthesized using the methods outlined below and in the Examples section, together with methods known in the art of organic synthetic organic chemistry, or variations thereof as appreciated by those skilled in the art. Preferred methods include, but are not limited to, those described below.

The novel compounds of formula (I) may be prepared using the reactions and techniques described in this section. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature duration of experiment and work-up procedures, are chosen to be conditions of standard for that reaction, which should be readily recognized by one skilled in the art. It is understood by one skilled in the art of organic synthesis that the functionality present on various portions of the educt molecule must be compatible with the reagents and reactions proposed. Not all molecules of formula (I) falling into a given class may be compatible with some of the reaction conditions required in some of the methods described. Such restrictions to the substituents which are compatible with the reaction conditions will be readily apparent to one skilled in the art and alternative methods can be used.

Compounds of general formula (I), in which r is 0 or X is —CH₂— can be prepared from an isatin-derivative by reaction with a Grignard-reagent or an organolithium reagent to form tertiary alcohols of general formula (II), which are subsequently allowed to react with phenol in a Friedel-Craft reaction in the presence of an acid, e.g. p-toluenesulphonic acid (p-TSA).

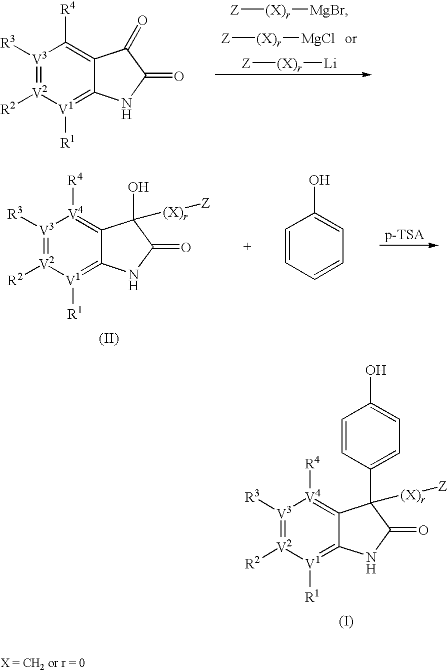

Isatin derivatives are either commercially available or can be prepared as described in the literature (e.g. Stolle: J. Prakt. Chem. (1922), 105, 137; Sandmeyer: Helv. Chim. Acta (1919), 2, 234; Shvedov et al.: Chem. Heterocycl. Comp. Engl. Transl. (1975). 11, 666; Hewawasam and Maenwell: Tet. Lett. (1994). 35, 7303; Rivalle and Bisagani: J. Heterocycl. Chem. (1997), 34, 441; Tatsugi et al.: ARKIVOC (2001), 67; Silva et al.: J. Braz. Chem. Soc. (2001), 12, 273).

Compounds (I) according to the present invention in which X is —CH₂— can also be prepared from tertiary alcohols of general formula (II), in which r is 0 and Z is a protected p-hydroxyphenyl (IIa), in which Pg is a protecting group (e.g. methyl, t-butyl, benzyl, triisopropylsilyl or other silyl protecting groups, tetrahydropyranyl, acetyl, benzoyl etc.), by dehydroxylation to yield deoxygenated intermediates of general formula (III), which are subsequently treated with a base (e.g. n-butyllithium and N,N,N,N-tetramethylehylenediamine) and an alkylating agent such as an alkylhalide to yield compounds of general formula (IV), followed by deprotection to yield compounds of general formula (I).

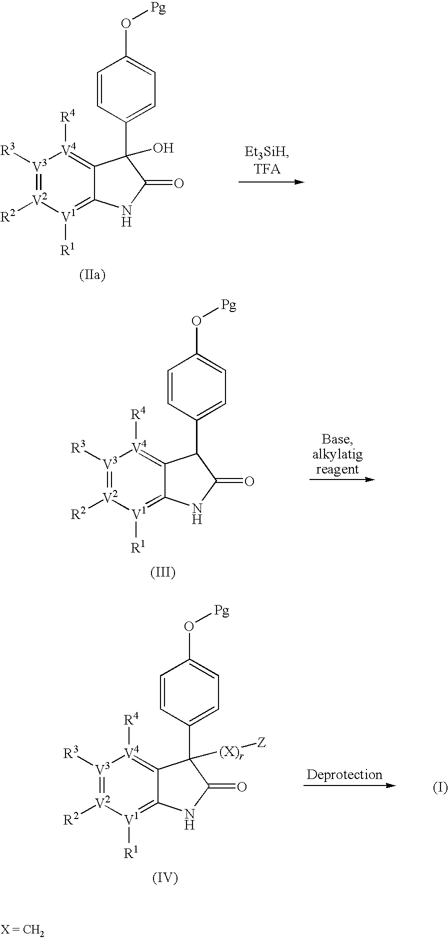

Compounds (I) according to the present invention, in which X is —NR⁵—, —O— or —S— can be prepared from tertiary alcohols of general formula (IIa), by conversion of the alcohol into a leaving group such as the chloro-compounds of general formula (V) and subsequent reaction with an amine, alcohol or thiol in the presence of a base, such as for instance diisopropylethylamine or sodium hydride, to yield intermediates of general formula (VI), and subsequent removal of the protecting group.

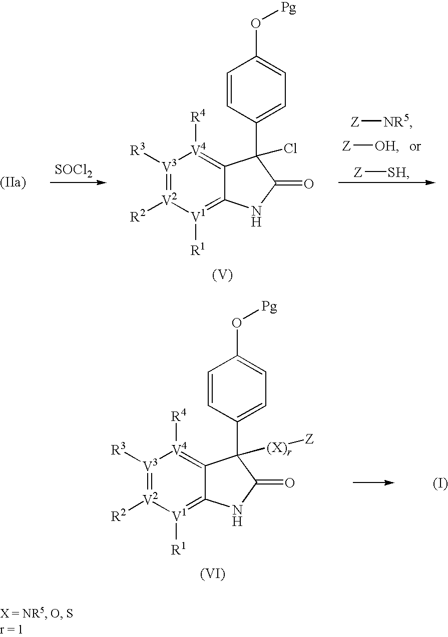

Compounds (I) according to the present invention in which X is —S(O)— or —S(O)₂— can be prepared from compounds of general formula (I) in which X is —S— by oxidation, e.g. by use of m-chloroperbenzoic acid in equimolar amount or excess, respectively.

Compounds (I) according to the present invention in which r is 0 and Z is imidazol attached via nitrogen can be prepared from tertiary alcohols (IIa) by reaction with 1,1′-carbonyldiimidazole to yield intermediates of general formula (VII) and subsequent removal of the protecting group.

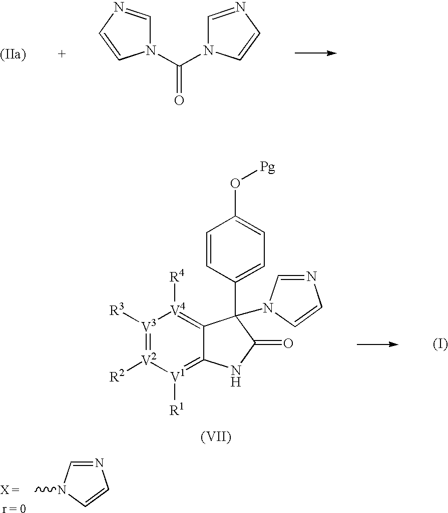

Compounds (I) according to the present invention in which X═CH₂— and Z is C₁₋₁₂-alkylcarbonyl, arylcarbonyl and heteroarylcarbonyl can be prepared from isatin derivatives in a Knoevenagel condensation with the corresponding ketones to yield intermediates of general formula (IIb), which are subsequently allowed to react with phenol in a Friedel-Craft reaction in the presence of an acid, e.g. p-TSA.

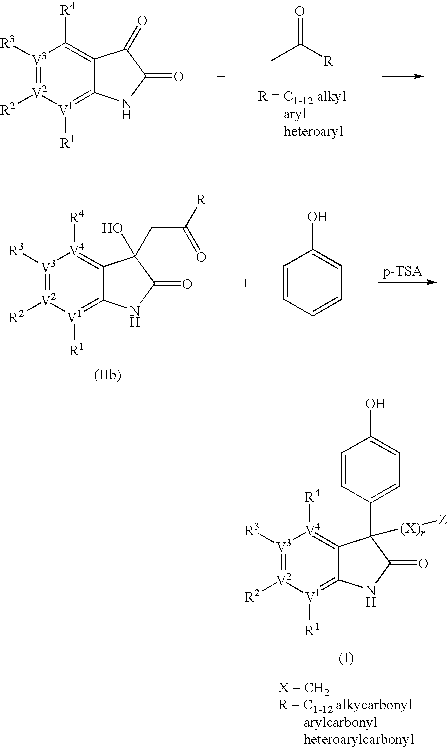

Compounds (I) according to the present invention which are racemates, can be resolved into the enantiomers by purification on a chiral column, e.g. Daicel Chiralcel-OD.

### Medical Uses

The compounds of the general formulae (I) and (Ia) are believed to be particularly useful in the treatment of cancer. The term cancer is typically describing cell growth not under strict control. In one embodiment of the invention, treatment of cancers in which inhibition of protein synthesis and/or inhibition of activation of the mTOR pathway is an effective method for reducing cell growth. Examples of such cancers include, but are not limited to, breast cancer, renal cancer, multiple myeloma, leukemia, glioblastoma, rhabdomyosarcoma, prostate, soft tissue sarcoma, colorectal sarcoma, gastric carcinoma, head and neck squamous cell carcinoma, uterine, cervical, melanoma, lymphoma, and pancreatic cancer.

Any type of cell may be treated, including but not limited to, lung, gastrointestinal (including e.g. bowel, colon), breast (mammary), ovarian, prostate, liver (hepatic), kidney (renal), bladder, pancreas, brain and skin.

Hence, the present invention generally provides a compound of the general formula (I) or (Ia) as defined herein for use as a medicament; more particular, the use of a compound of the general formula (I) or (Ia) as defined herein for the preparation of a medicament for the treatment of cancer in a mammal. Such medicaments may further comprise one or more other chemotherapeutic agents.

Moreover, the present invention provides a method of treating a mammal suffering from or being susceptible to cancer, the method comprising administering to the mammal a therapeutically effective amount of a compound of the general formula (I) or (Ia) as defined herein.

### Formulation of Pharmaceutical Compositions

The compounds of the general formulae (I) and (Ia) are suitably formulated in a pharmaceutical composition so as to suit the desirable route of administration.

The administration route of the compounds may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question; particularly the choice of administration route depends on the physico-chemical properties of the compound together with the age and weight of the patient and on the particular disease or condition and the severity of the same.

The compounds may be contained in any appropriate amount in a pharmaceutical composition, and are generally contained in an amount of about 1-95%, e.g. 1-10%, by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier or excipient. Pharmaceutically acceptable carriers or excipients are those known by the person skilled in the art. Formation of suitable salts of the compounds of the Formulae (I) and (Ia) will also be evident in view of the before-mentioned.

Thus, the present invention provides in a further aspect a pharmaceutical composition comprising a compound of the general Formula (I) or (Ia) in combination with a pharmaceutically acceptable carrier.

Pharmaceutical compositions according to the present invention may be formulated to release the active compound substantially immediately upon administration or at any substantially predetermined time or time period after administration. The latter type of compositions is generally known as controlled release formulations.

In the present context, the term “controlled release formulation” embraces i) formulations which create a substantially constant concentration of the drug within the body over an extended period of time, ii) formulations which after a predetermined lag time create a substantially constant concentration of the drug within the body over an extended period of time, iii) formulations which sustain drug action during a predetermined time period by maintaining a relatively, constant, effective drug level in the body with concomitant minimization of undesirable side effects associated with fluctuations in the plasma level of the active drug substance (saw-tooth kinetic pattern), iv) formulations which attempt to localize drug action by, e.g., spatial placement of a controlled release composition adjacent to or in the diseased tissue or organ, v) formulations which attempt to target drug action by using carriers or chemical derivatives to deliver the drug to a particular target cell type.

Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

Preparation of solid dosage forms for oral use, controlled release oral dosage forms, fluid liquid compositions, parenteral compositions, controlled release parenteral compositions, rectal compositions, nasal compositions, percutaneous and topical compositions, controlled release percutaneous and topical compositions, and compositions for administration to the eye will be well-known to those skilled in the art of pharmaceutical formulation. Specific formulations can be found in “Remington's Pharmaceutical Sciences”.

Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The pharmaceutical compositions may also be emulsions of the compound(s) and a lipid forming a micellular emulsion.

For parenteral, subcutaneous, intradermal or topical administration the pharmaceutical composition may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

### Dosages

In one embodiment, the pharmaceutical composition is in unit dosage form. In such embodiments, each unit dosage form typically comprises 0.1-500 mg, such as 0.1-200 mg, e.g. 0.1-100 mg, of the compound.

More generally, the compound are preferably administered in an amount of about 0.1-250 mg per kg body weight per day, such as about 0.5-100 mg per kg body weight per day.

For compositions adapted for oral administration for systemic use, the dosage is normally 0.5 mg to 1 g per dose administered 1-4 times daily for 1 week to 12 months depending on the disease to be treated.

The dosage for oral administration of the composition in order to prevent diseases or conditions is normally 1 mg to 100 mg per kg body weight per day. The dosage may be administered once or twice daily for a period starting 1 week before the exposure to the disease until 4 weeks after the exposure.

For compositions adapted for rectal use for preventing diseases, a somewhat higher amount of the compound is usually preferred, i.e. from approximately 1 mg to 100 mg per kg body weight per day.

For parenteral administration, a dose of about 0.1 mg to about 100 mg per kg body weight per day is convenient. For intravenous administration, a dose of about 0.1 mg to about 20 mg per kg body weight per day administered for 1 day to 3 months is convenient. For intraarticular administration, a dose of about 0.1 mg to about 50 mg per kg body weight per day is usually preferable. For parenteral administration in general, a solution in an aqueous medium of 0.5-2% or more of the active ingredients may be employed.

For topical administration on the skin, a dose of about 1 mg to about 5 g administered 1-10 times daily for 1 week to 12 months is usually preferable.

### Combination Treatment

In an intriguing embodiment of the present invention, the compound of the general formula (I) or (Ia) is used therapeutically in combination with one or more other chemotherapeutic agents. Examples of such chemotherapeutic agents are those selected from daunorubicin, docetaxel, prednisone, dexamethasone, decadron, altretamine, amifostine, aminoglutethimide, dactinomycin, anastrozole, asparaginase, bicalutamide, bleomycin, busulfan, carboplatin, carmustine, chlorambucil, chlorodeoxyadenosine, cisplatin, cytosine arabinoside, dacarbazine, doxorubicin, epirubicin, estramustine, diethylstilbestrol, fludarabine, flutamide, 5-fluorouracil, gemcitabine, goserelin, idarubicin, irinotecan, levamisole, lomustine, mechlorathamine, alkeran, mercaptopurine, taxol (e.g. paclitaxel). In particular, the further chemotherapeutic agent is selected from taxanes such as Taxol, Paclitaxel and Docetaxel.

Thus, with respect to the use and the method of treatment defined herein, the medicament may further comprise one or more other chemotherapeutic agents.

### EXAMPLES

### General Procedures

For nuclear magnetic resonance ¹H-NMR spectra (300 MHz) and ¹³C-NMR (75.6) chemical shift values (δ) (in ppm) are quoted, unless otherwise specified, for deuterochloroform solutions relative to tetramethylsilane (δ=0.0) or chloroform (δ=7.25) or deuterochloroform (δ=76.81 for ¹³C-NMR) standards. The value of a multiplet, either defined (doublet (d), triplet (t) quartet (q)) or not (m) at the approximate mid point is given unless a range is quoted. (bs) indicates a broad singlet.

MS was performed using a Micromass LCT with an AP-ESI-probe or LC-MS using a Bruker Esquire 3000+ESI Iontrap with an Agilent 1200 HPLC-system.

The organic solvents used were anhydrous.

The following abbreviations have been used throughout:

DCM dichloromethane
DMAP N,N dimethylaminopyridine
EtOAc ethyl acetate
MS mass spectroscopy
NMR nuclear magnetic resonance
n-BuLi n-butyl lithium
rt room temperature
p-TSA para-toluenesulphonic acid
TFA trifluoroacetic acid
TLC thin layer chromatography

### TMEDA N,N,N,N-tetramethylenediamine

### General Procedure 1: Grignard Reaction to Form Tertiary Alcohols of General Formula (II).

To a stirred solution of isatin derivative in dry THF under nitrogen at −78° C. was added 3 eq. of Grignard reagent or 3 eq. of freshly prepared solution of organolithium reagent. After 30 min, the dry-ice bath was removed and the reaction was left to reach room temperature over 4 to 14 hours. Excess Grignard reagent was quenched with water, and the reaction mixture was acidified with 1N HCl or saturated NH₄Cl-solution, extracted with EtOAc (2×), dried over Mg₂SO₄, filtered and concentrated. The residue was purified by chromatography (1% methanol in DCM or mixtures of petroleum ether and EtOAc) to afford racemic compounds of general formula II).

### General Procedure 2: Friedel-Craft Reaction to Form Compounds of General Formula (I).

To a solution of tertiary alcohol of general formula (II) in dichloroethane was added phenol (5 eq.) and p-TSA (7.5 eq.). The reaction mixture was heated to 90° C. for 2-4 hours and then cooled to room temperature. The solid (mainly p-TSA) was filtered off and washed with dichloroethane or DCM. The solution was concentrated and the residue was purified by chromatography (1% methanol in DCM or mixtures of petroleum ether and EtOAc) to afford racemic compounds of general formula (I).

### General Procedure 3: Dehydroxylation of Tertiary Alcohols (IIa) to Yield Deoxygenated Intermediates (III).

A mixture of tertiary alcohol (IIa), Et₃SiH (3 eq.) and TFA were heated to 100° C. in a sealed tube for 1-3 days until the deoxygenation was complete. Excess Et₃SiH and TFA were evaporated, and the residue was purified by chromatography (1% methanol in DCM or mixtures of petroleum ether and EtOAc) to afford racemic compounds of general formula (III).

### General Procedure 4: Alkylation of Compounds of General Formula (III) to Yield Compounds of General Formula (IV).

Compound of general formula (III) was dissolved in dry THF under nitrogen, TMEDA (2.2 eq.) was added and the mixture was cooled to −78° C. 1.6 M n-BuLi solution (2.2 eq.) was added dropwise and the mixture stirred at −78° C. for 0.5-1 hour. The alkylating agent (2.2 eq.) was then added and the reaction mixture gradually allowed to reach room temperature. After 3-8 hours the mixture was quenched with water, extracted with EtOAc (2×), dried over Mg₂SO₄, filtered and concentrated. The residue was purified by chromatography (1% methanol in DCM or mixtures of petroleum ether and EtOAc) to afford racemic compounds of general formula (IV).

### General Procedure 5: Demethylation of Compounds of General Formula (IV) to Yield Compounds of General Formula (I).

Compound of general formula (IV), in which the protecting group is a methyl group, was dissolved in DCM under nitrogen, cooled to −78° C. and BBr₃-solution (1.0 M, 1.5 eq.) was added dropwise with stirring. The reaction mixture was gradually allowed to reach room temperature. After 4-18 hours the mixture was quenched with water, extracted with Et₂O (2×), dried over Mg₂SO₄, filtered and concentrated. The residue was purified by chromatography (1% methanol in DCM or mixtures of petroleum ether and EtOAc) to afford racemic compounds of general formula (I).

### General Procedure 6: HPLC Purification on Daicel Chiralcel-OD 250×20 mm ID 5 Micron to Yield Pure Enantiomers of General Formula (I).

Racemic compound of general formula (I) was dissolved in ethanol or ethanol/heptane mixtures and purified by HPLC on Daicel Chiralcel-OD 250×20 mm ID 5 micron to yield pure enantiomers of general formula (I).

### Preparations

### Preparation 1: 3-ethynyl-6,7-difluoro-3-hydroxyindoline-2-one (compound 1)

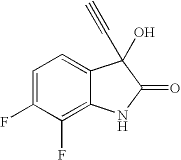

General procedure 1. Starting materials: 6,7-difluoroindoline-2,3-dione and ethynylmagnesium chloride. ¹H-NMR (DMSO-d₆) δ 11.33 (bs), 7.3-7.2 (m, 1H), 7.15 (s, 1H), 7.1-7.0 (m, 1H), 3.68 (s, 1H).

### Preparation 2: 6,7-difluoro-3-hydroxy-3-(4-methoxyphenyl)indolin-2-one (compound 2)

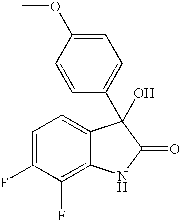

General procedure 1. Starting materials: 6,7-difluoroindoline-2,3-dione and 4-methoxymagnesium bromide. ¹H-NMR (DMSO-d₆) δ 11.12 (bs), 7.25-7.15 (d, 2H), 7.1-6.9 (m, 4H), 6.71 (s, 1H), 3.73 (s, 3H).

### Preparation 3: 6,7-difluoro-3-(4-methoxyphenyl)indolin-2-one (compound 3)

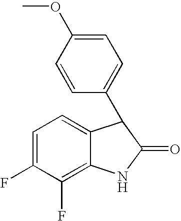

General procedure 3. Starting materials: Starting materials: 6,7-difluoro-3-hydroxy-3-(4-methoxyphenyl)indolin-2-one (compound 2). ¹H-NMR (CDCl₃) δ 8.08 (bs, 1H), 7.2-7.1 (d, 2H), 7.0-6.8 (m, 4H), 4.61 (s, 1H), 3.82 (s, 3H).

### Preparation 4: 3-benzyl-6,7-difluoro-3-(4-methoxyphenyl)indolin-2-one (compound 4)

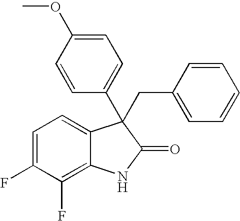

General procedure 4. Starting materials: Compound 3 and benzyl bromide. ¹H-NMR (CDCl₃) δ 7.69 (bs, 1H), 7.3-7.2 (d, 2H), 7.05-6.9 (m, 3H), 6.8-6.6 (m, 6H), 3.67 (s, 3H), 3.55 (d, 1H), 3.28 (d, 1H).

### Preparation 5: 6,7-difluoro-3-hydroxy-3-methylindolin-2-one (compound 5)

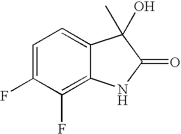

General procedure 1. Starting materials: 6,7-difluoroindoline-2,3-dione and methylmagnesium bromide. ¹H-NMR (DMSO-d₆) δ 10.99 (bs, 1H), 7.15 (m, 1H), 5.98 (m, 1H), 6.03 (s, 1H), 1.37 (s, 3H).

### Preparation 6: 3-cyclopentyl-6,7-difluoro-3-hydroxyindolin-2-one (compound 6)

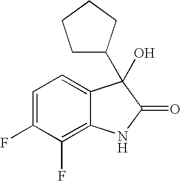

General procedure 1. Starting materials: 6,7-difluoroindoline-2,3-dione and cyclopentylmagnesium bromide. ¹H-NMR (DMSO-d₆) δ 10.98 (bs, 1H), 7.15 (m, 1H), 5.94 (m, 1H), 2.31 (m, 1H), 1.7-1.3 (m, 7H), 1.15 (m, 1H).

### Preparation 7: 3-(cyclohexylmethyl)-6,7-difluoro-3-hydroxyindolin-2-one (compound 7)

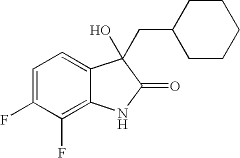

General procedure 1. Starting materials: 6,7-difluoroindoline-2,3-dione and cyclohexylmethylmagnesium bromide. ¹H-NMR (DMSO-d₆) δ 11.01 (bs, 1H), 5.96 (m, 1H), 6.99 (m, 1H), 1.74 (d, 2H), 1.6-1.3 (m, 5H), 1.1-0.7 (m, 6H).

### Preparation 8: 6,7-difluoro-3-hydroxy-3-(pyridin-4-yl)indolin-2-one (compound 8)

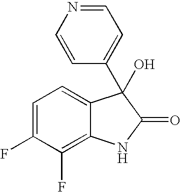

General procedure 1. Starting materials: 6,7-difluoroindoline-2,3-dione and pyridine-4-ylmagnesium bromide. ¹H-NMR (DMSO-d₆) δ 11.37 (bs, 1H), 8.53 (m, 2H), 7.27 (m, 2H), 7.08 (s, 1H), 7.06-6.9 (m, 2H).

### Preparation 9: 6,7-difluoro-3-hydroxy-3-isopropylindolin-2-one (compound 9)

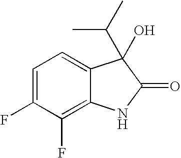

General procedure 1. Starting materials: 6,7-difluoroindoline-2,3-dione and isopropylmagnesium bromide. ¹H-NMR (DMSO-d₆) δ 11.00 (bs, 1H), 7.1-6.9 (m, 2H), 5.99 (s, 1H), 2.08 (m, 1H), 0.95 (d, 3H), 0.66 (d, 3H).

### Preparation 10: 6,7-difluoro-3-hydroxy-3-(thiophen-2-yl)indolin-2-one (compound 10)

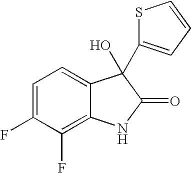

General procedure 1. Starting materials: 6,7-difluoroindoline-2,3-dione and thiophen-2-ylmagnesium bromide. ¹H-NMR (DMSO-d₆) δ 11.23 (bs, 1H), 7.91 (d, 1H), 7.52 (dd, 1H), 7.16 (m, 1H), 7.06 (m, 1H) 7.05 (s, 1H), 6.95 (dd, 1H), 6.71 (dd, 1H).

### Preparation 11: 3-butyl-6,7-difluoro-3-hydroxyindolin-2-one (compound 11)

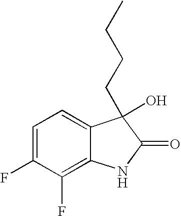

General procedure 1. Starting materials: 6,7-difluoroindoline-2,3-dione and n-butylylmagnesium bromide. ¹H-NMR (DMSO-d₆) δ 11.00 (bs, 1H), 7.09 (m, 1H), 6.98 (s, 1H), 6.01 (m, 1H), 1.77 (m, 2H), 1.18 (m, 2H), 1.08-0.84 (m, 2H), 0.78 (t, 3H).

### Preparation 12: 3-cyclohexyl-6,7-difluoro-3-hydroxyindolin-2-one (compound 12)

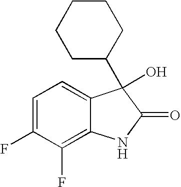

General procedure 1. Starting materials: 6,7-difluoroindoline-2,3-dione and cyclohexylmagnesium bromide. ¹H-NMR (DMSO-d₆) δ 10.98 (bs, 1H), 7.06 (m, 1H), 6.97 (m, 1H), 5.94 (s, 1H), 0.75-0.6 (m, 6H), 1.2-0.9 (m, 4H), 1.08-0.84 (m, 1H).

### Preparation 13: 6,7-difluoro-3-hydroxy-3-propylindolin-2-one (compound 13)

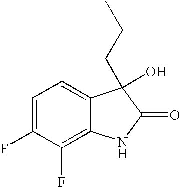

General procedure 1. Starting materials: 6,7-difluoroindoline-2,3-dione and n-propylmagnesium bromide. ¹H-NMR (DMSO-d₆) δ 11.00 (bs, 1H), 7.09 (m, 1H), 6.98 (m, 1H), 6.01 (s, 1H), 1.75 (m, 2H), 1.1-0.9 (m, 2H), 0.78 (t, 3H).

### Preparation 14: 6,7-difluoro-3-hydroxy-3-pentylindolin-2-one (compound 14)

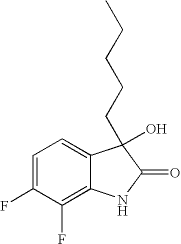

General procedure 1. Starting materials: 6,7-difluoroindoline-2,3-dione and n-pentylmagnesium bromide. ¹H-NMR (DMSO-d₆) δ 11.00 (bs, 1H), 7.09 (m, 1H), 6.98 (m, 1H), 6.01 (s, 1H), 1.76 (m, 2H), 1.25-0.9 (m, 2H), 0.78 (t, 3H).

### Preparation 15: 6,7-difluoro-3-hydroxy-3-(thiophen-3-yl)indolin-2-one (compound 15)

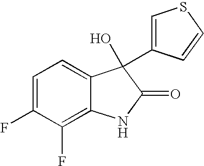

General procedure 1. Starting materials: 6,7-difluoroindoline-2,3-dione and thiophen-3-ylmagnesium bromide. ¹H-NMR (DMSO-d₆) δ 11.16 (bs, 1H), 7.50 dd, 1H), 7.24 (dd, 1H), 7.15-6.9 (m, 3H), 6.76 (s, 1H).

### Preparation 16: 6,7-difluoro-3-hydroxy-3-(pyridin-3-yl)indolin-2-one (compound 16)

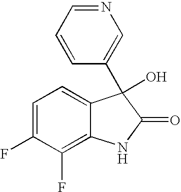

General procedure 1. Starting materials: 6,7-difluoroindoline-2,3-dione and pyridin-3-ylmagnesium bromide. ¹H-NMR (CDCl₃) δ 8.61 (bs, 1H), 8.37 (1H), 8.15 (1H, bs), 7.69 (1H), 7.16 (1H), 6.89 (m, 1H), 6.73 (m, 1H), 5.23 (s, 1H).

### Preparation 17: 3-(but-en-2-yl)-6,7-difluoro-3-hydroxyindolin-2-one (compound 17, 2 diastereoisomers)

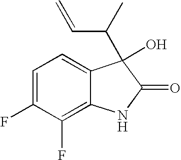

General procedure 1. Starting materials: 6,7-difluoroindoline-2,3-dione and but-3-en-2-ylmagnesium chloride.

### Preparation 18: 3-sec-butyl-6,7-difluoro-3-hydroxyindolin-2-one (compound 18, 2 diastereoisomers)

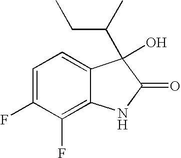

General procedure 1. Starting materials: 6,7-difluoroindoline-2,3-dione and sec-butylmagnesium chloride.

### Preparation 19: 3-cycloheptyl-6,7-difluoro-3-hydroxyindolin-2-one (compound 19)

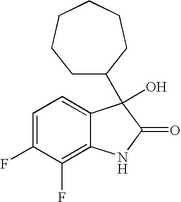

General procedure 1. Starting materials: 6,7-difluoroindoline-2,3-dione and cycloheptylmagnesium bromide.

### Preparation 20: 3-(1-(benzyloxy)-1H-pyrazol-4-yl)-6,7-difluoro-3-hydroxyindolin-2-one (compound 20)

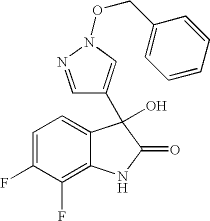

General procedure 1. Starting materials: 6,7-difluoroindoline-2,3-dione and (1-(benzyloxy)-1H-pyrazol-4-yl)magnesium bromide (J. Org. Chem. (1999) 64, 4196-4198). ¹H-NMR (CDCl₃) δ 9.01 (bs, 1H), 7.4-7.2 (m, 7H), 7.04 (m, 2H), 6.84 (m, 1H), 5.19 (s, 2H).

### Preparation 21: 3-cyclohexyl-3-hydroxy-7-(trifluoromethyl)indolin-2-one (compound 21)

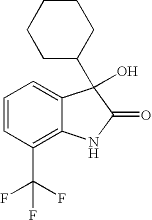

General procedure 1. Starting materials: 7-(trifluoromethyl)indoline-2,3-dione and cyclohexylmagnesium chloride. ¹H-NMR (CDCl₃) δ 7.86 (bs, 1H), 7.52 (dd, 2H), 7.18 (d, 1H), 2.92 (s, 1H), 2.0-1.5 (m, 6H), 1.40-1.0 (m, 4H), 0.86 (m, 1H).

### Preparation 22: 3-(3,4-difluorophenyl)-6,7-difluoro-3-hydroxyindolin-2-one (compound 22)

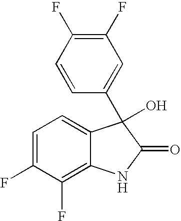

General procedure 1. Starting materials: 6,7-difluoroindoline-2,3-dione and (3,4-difluorophenyl)magnesium bromide. ¹H-NMR (DMSO-d₆) δ 13.74 (bs, 1H), 8.1 (m, 2H), 7.73 (m, 1H), 7.49 (m, 2H), 6.99 (s, 1H).

### Preparation 23: 6,7-difluoro-3-(3-fluoro-4-methylphenyl)-3-hydroxyindolin-2-one (compound 23)

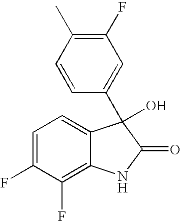

General procedure 1. Starting materials: 6,7-difluoroindoline-2,3-dione and (3-fluoro-4-methylphenyl)magnesium bromide. ¹H-NMR (DMSO-d₆) δ 11.20 (bs, 1H), 7.21 (m, 1H), 7.11 (m, 1H), 7.03-6.78 (m, 6H), 2.19 (s, 3H).

### Preparation 24: 6-chloro-3-cyclohexyl-3-hydroxy-7-methylindolin-2-one (compound 24)

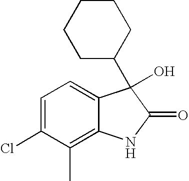

General procedure 1. Starting materials: 6-chloro-7-methylindoline-2,3-dione and cyclohexylmagnesium bromide. ¹H-NMR (DMSO-d₆) δ 10.47 (bs, 1H), 7.04 (m, 2H), 5.79 (s, 1H), 2.21 (s, 3H), 1.9-1.4 (m, 6H), 1.2-0.85 (m, 4H), 0.65 (m, 1H).

### Preparation 25: 3-cyclohexyl-3-hydroxy-6,7-dimethylindolin-2-one (compound 25)

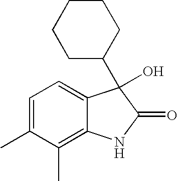

General procedure 1. Starting materials: 6,7-dimethylindoline-2,3-dione and cyclohexylmagnesium bromide. ¹H-NMR (DMSO-d₆) δ 10.14 (bs, 1H), 6.93 (d, 1H), 6.6 (d, 1H), 5.56 (s, 1H), 2.19 (s, 3H) 2.08 (s, 3H), 1.9-1.4 (m, 6H), 1.2-0.85 (m, 4H), 0.63 (m, 1H).

### Preparation 26: 3-(cyclopentylmethyl)-6,7-difluoro-3-hydroxyindolin-2-one (compound 26)

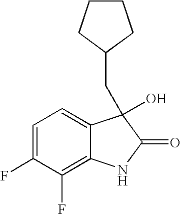

General procedure 1. Starting materials: 6,7-difluoroindoline-2,3-dione and (cyclopentylmethyl)magnesium bromide. ¹H-NMR (DMSO-d₆) δ 11.01 (bs, 1H), 7.10 (m, 1H), 6.98 (m, 1H), 5.98 (s, 1H), 1.93 (m, 2H), 1.65-1.2 (m, 7H), 1.0 (m, 1H), 0.84 (m, 1H).

### Preparation 27: 6,7-difluoro-3-(4-hydroxy-3-methylphenyl)-3-(4-methoxyphenyl)indolin-2-one (compound 27)

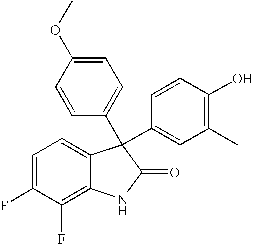

General procedure 2 using o-cresol instead of phenol. Starting materials: compound 2. ¹H-NMR (CDCl₃) δ 7.78 (bs, 1H), 7.08 (d, 2H), 6.95-6.7 (m, 6H), 6.59 (d, 2H), 3.71 (s, 3H), 2.10 (s, 3H).

### Preparation 28: 6,7-difluoro-3-(4-hydroxy-2-methylphenyl)-3-(4-methoxyphenyl)indolin-2-one (compound 28)

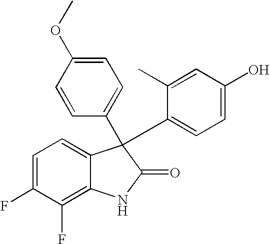

General procedure 2 using m-cresol instead of phenol. Starting materials: compound 2. ¹H-NMR (DMSO-d₆) δ 11.05 (bs, 1H), 9.47 (bs, 1H), 7.22 (d, 2H), 6.93 (m, 3H), 6.51 (m, 3H), 3.75 (s, 3H), 2.17 (s, 3H).

### Preparation 29: 3-cyclooctyl-6,7-difluoro-3-hydroxyindolin-2-one (compound 29)

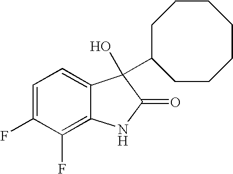

General procedure 1. Starting materials: 6,7-difluoroindoline-2,3-dione and cyclooctylmagnesium bromide. ¹H-NMR (DMSO-d₆) δ 11.02 (bs, 1H), 7.10 (m, 1H), 6.95 (m, 1H), 2.04 (m, 1H), 1.76-1.20 (m, 13H), 0.86 (m, 1H).

### Preparation 30: 6,7-difluoro-3-hydroxy-3-(naphthalene-1-yl)indolin-2-one (compound 30)

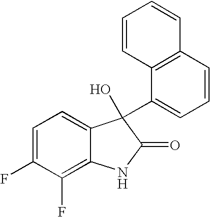

General procedure 1. Starting materials: 6,7-difluoroindoline-2,3-dione and naphthalene-1-ylmagnesium bromide. ¹H-NMR (CDCl₃) δ 8.23 (bs, 1H), 7.98-7.8 (m, 4H), 7.55-7.4 (m, 3H), 7.28 (s, 1H), 6.96 (m, 1H), 6.76 (m, 1H).

### Preparation 31: 6,7-difluoro-3-hydroxy-3-(naphthalene-2-yl)indolin-2-one (compound 31)

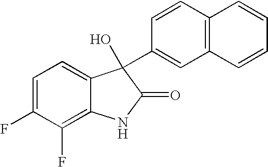

General procedure 1. Starting materials: 6,7-difluoroindoline-2,3-dione and naphthalene-2-ylmagnesium bromide. ¹H-NMR (DMSO-d₆) δ 11.29 (bs, 1H), 8.0-7.8 (m, 4H), 7.52 (m, 2H), 7.36 (dd, 1H), 7.06-6.95 (m, 3H).

### Preparation 32: 3-cycloheptyl-7-fluoro-3-hydroxy-6-methylindolin-2-one (compound 32)

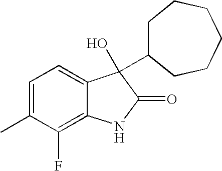

General procedure 1. Starting materials: 7-fluoro-6-methylindoline-2,3-dione and cycloheptylmagnesium bromide. ¹H-NMR (CDCl₃) δ 8.38 (bs, 1H), 7.07 (d, 1H), 6.87 (t, 1H), 5.40 (bs, 1H), 2.28 (m, 4H), 2.15-1.3 (m, 11H), 0.90 (m, 1H).

### Preparation 33: 3-cyclohexyl-7-fluoro-3-hydroxy-6-methylindolin-2-one (compound 33)

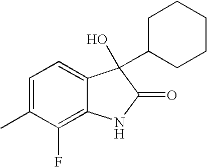

General procedure 1. Starting materials: 7-fluoro-6-methylindoline-2,3-dione and cyclohexylmagnesium bromide. ¹H-NMR (DMSO-d₆) δ 10.62 (bs, 1H), 6.96 (d, 1H), 6.29 (t, 1H), 5.80 (s, 1H), 2.22 (d, 3H), 1.9-1.4 (m, 6H), 1.25-0.85 (m, 4H), 0.62 (m, 1H).

### Preparation 34: 3-tert-butyl-6,7-difluoro-3-hydroxyindolin-2-one (compound 34)

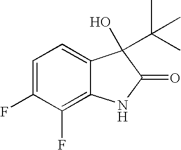

General procedure 1. Starting materials: 6,7-difluoro-indoline-2,3-dione and tert-butylmagnesium chloride. ¹H-NMR (DMSO-d₆) δ 10.97 (bs, 1H), 7.14 (m, 1H), 7.00 (m, 1H), 5.93 (s, 1H), 1.00 (s, 3H).

### Preparation 35: 6,7-difluoro-3-hydroxy-3-tert-pentylindolin-2-one (compound 35)

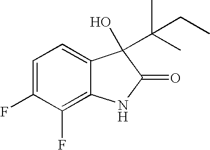

General procedure 1. Starting materials: 6,7-difluoro-indoline-2,3-dione and 1,1-dimethylpropylbutylmagnesium chloride. ¹H-NMR (DMSO-d₆) δ 10.97 (bs, 1H), 7.07 (m, 1H), 6.94 (m, 1H), 5.87 (s, 1H), 1.3 (m, 2H), 0.92 (s, 6H), 0.75 (t, 3H).

### Preparation 36: 3-cyclopentyl-6-fluoro-3-hydroxy-7-methylindolin-2-one (compound 36)

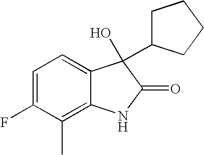

General procedure 1. Starting materials: 6-fluoro-7-methylindoline-2,3-dione and cyclopentylmagnesium bromide. ¹H-NMR (CDCl₃) δ 8.72 (bs, 1H), 7.05 (m, 1H), 6.61 (t, 1H), 2.38 (m, 1H), 2.09 (s, 3H), 1.8-1.3 (m, 7H), 1.07 (m, 1H).

### Preparation 37: 3-cyclohexyl-6-fluoro-3-hydroxy-7-methylindolin-2-one (compound 37)

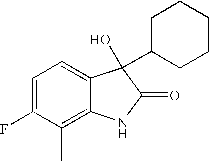

General procedure 1. Starting materials: 6-fluoro-7-methylindoline-2,3-dione and cyclohexylmagnesium chloride. ¹H-NMR (CDCl₃) δ 9.32 (bs, 1H), 7.14 (m, 1H), 6.73 (t, 1H), 2.17 (s, 3H), 2.05-0.95 (m, 10H), 0.78 (m, 1H).

### Preparation 38: 6-fluoro-3-hydroxy-7-methyl-3-pentylindolin-2-one (compound 38)

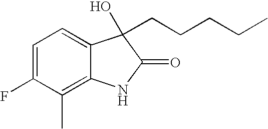

General procedure 1. Starting materials: 6-fluoro-7-methylindoline-2,3-dione and n-pentylmagnesium bromide. ¹H-NMR (DMSO-d₆) δ 10.47 (bs, 1H), 7.08 (m, 1H), 6.73 (m, 1H), 5.81 (bs, 1H), 2.12 (s, 3H), 1.17 (m, 2H), 1.4-0.8 (m, 6H), 0.78 (t, 3H).

### Preparation 39: 3-cycloheptyl-6-fluoro-3-hydroxy-7-methylindolin-2-one (compound 39)

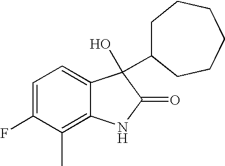

General procedure 1. Starting materials: 6-fluoro-7-methylindoline-2,3-dione and cycloheptylmagnesium bromide. ¹H-NMR (CDCl₃) δ 8.58 (bs, 1H), 7.18 (m, 1H), 6.38 (m, 1H), 2.85 (m, 1H), 2.20 (s, 3H), 2.3-1.3 (m, 11H), 0.90 (m, 1H).

### Preparation 40: 3-cycloheptyl-3-hydroxy-7-(trifluoromethyl)indolin-2-one (compound 40)

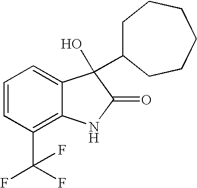

General procedure 1. Starting materials: 7-(trifluoromethyl)indoline-2,3-dione and cycloheptylmagnesium bromide. ¹H-NMR (DMSO-d₆) δ 10.73 (bs, 1H), 7.51 (m, 2H), 7.10 (t, 1H), 6.04 (s, 1H), 2.2-1.1 (m, 12H), 0.80 (m, 1H).

### Preparation 41: 3-cycloheptyl-3-hydroxy-6,7-dimethylindolin-2-one (compound 41)

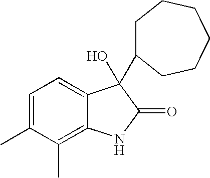

General procedure 1. Starting materials: 6,7-dimethylindoline-2,3-dione and cycloheptylmagnesium bromide.

### Preparation 42: 6-chloro-3-cycloheptyl-3-hydroxy-7-methylindolin-2-one (compound 42)

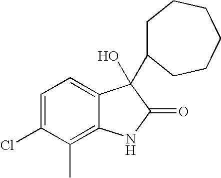

General procedure 1. Starting materials: 6-chloro-7-methylindoline-2,3-dione and cycloheptylmagnesium bromide. ¹H-NMR (DMSO-d₆) δ 10.49 (bs, 1H), 7.50 (d, 2H), 7.01 (d, 1H), 5.85 (s, 1H), 2.22 (s, 3H), 2.1-1.1 (m, 12H), 0.79 (m, 1H).

### Preparation 43: 3-cyclopentyl-3-hydroxy-6-methoxy-7-methylindolin-2-one (compound 43)

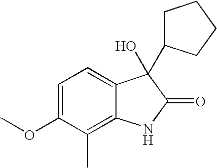

General procedure 1. Starting materials: 6-methoxy-7-methylindoline-2,3-dione and cyclopentylmagnesium bromide.

### Preparation 44: 3-cyclohexyl-3-hydroxy-6-methoxy-7-methylindolin-2-one (compound 44)

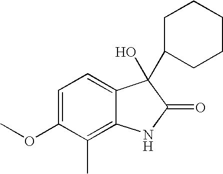

General procedure 1. Starting materials: 6-methoxy-7-methylindoline-2,3-dione and cyclohexylmagnesium chloride. ¹H-NMR (DMSO-d₆) δ 10.19 (bs, 1H), 6.99 (d, 1H), 6.51 (d, 1H), 5.52 (s, 1H), 3.76 (s, 3H), 2.01 (s, 3H), 1.95-1.4 (m, 6H), 1.25-0.85 (m, 4H), 0.63 (m, 1H).

### Preparation 45: 3-cycloheptyl-3-hydroxy-6-methoxy-7-methylindolin-2-one (compound 45)

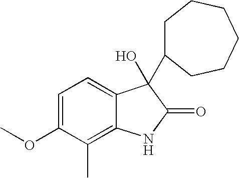

General procedure 1. Starting materials: 6-methoxy-7-methylindoline-2,3-dione and cycloheptylmagnesium bromide. ¹H-NMR (DMSO-d₆) δ 10.22 (bs, 1H), 7.01 (d, 1H), 6.50 (d, 1H), 5.59 (s, 1H), 3.76 (s, 3H), 2.01 (s, 3H), 2.2-1.2 (m, 13H), 0.75 (m, 1H).

### Preparation 46: 3-(4-(benzyloxy)phenyl)-3-hydroxy-7-(trifluoromethyl)indolin-2-one (compound 46)

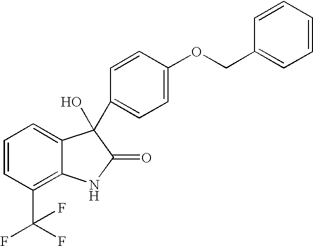

General procedure 1. Starting materials: 7-(trifluoromethyl)indoline-2,3-dione and (4-(benzyloxy)phenyl)magnesium bromide. ¹H-NMR (DMSO-d₆) δ 10.85 (bs, 1H), 7.55 (d, 1H), 7.5-7.28 (m, 6H), 7.18 (m, 3H), 6.98 (m, 2H), 6.75 (s, 1H), 5.08 (s, 2H).

### Preparation 47: 3-(4-(benzyloxy)phenyl)-3-(1H-imidazol-1-yl)-7-(trifluoromethyl)indolin-2-one (compound 47)

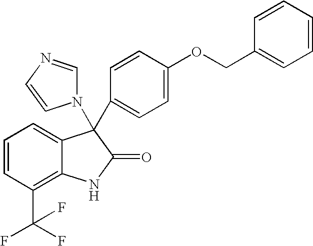

Imidazole (204 mg, 3 mmol) was dissolved in DCM (dried), cooled to 0° C., and thionylchloride (55 μl, 0.75 mmol) was added with stirring. After 30 minutes compound 46 (200 mg, 0.5 mmol) was added. After a further 2 h the reaction mixture was extracted with H₂O, brine, dried over Mg₂SO₄, filtered and concentrated. The residue was purified by chromatography (1% methanol in DCM) to afford compound 47. ¹H-NMR (DMSO-d₆) δ 11.59 (bs, 1H), 7.82 (d, 1H), 7.69 (d, 1H), 7.59 (s, 1H), 7.5-7.2 (m, 6H), 7.15-6.95 (m, 6H), 5.10 (s, 2H).

### Preparation 48: 3-(4-(benzyloxy)phenyl)-6,7-difluoro-3-hydroxyindolin-2-one (compound 48)

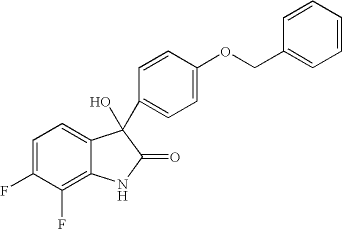

General procedure 1. Starting materials: 6,7-difluoromethylindoline-2,3-dione and (4-(benzyloxy)phenyl)magnesium bromide. ¹H-NMR (DMSO-d₆) δ 11.13 (bs, 1H), 7.5-7.28 (m, 5H), 7.19 (m, 2H), 7.05-6.90 (m, 4H), 6.72 (s, 1H), 5.08 (s, 2H).

### Preparation 49: 3-(4-(benzyloxy)phenyl)-6,7-difluoro-3-(1H-imidazol-1-yl)indolin-2-one (compound 49)

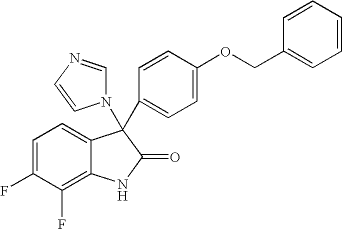

Imidazole (204 mg, 3 mmol) was dissolved in DCM (dried), cooled to 0° C., and thionylchloride (55 μl, 0.75 mmol) was added with stirring. After 30 minutes compound 48 (200 mg, 0.5 mmol) was added. After a further 2 h the reaction mixture was extracted with H₂O, brine, dried over Mg₂SO₄, filtered and concentrated. The residue was purified by chromatography (1% methanol in DCM) to afford compound 49. ¹H-NMR (CDCl₃) δ 8.94 (bs, 1H), 7.59 (s, 1H), 7.5-7.3 (m, 5H), 7.25-7.1 (m, 3H), 7.09-6.85 (m, 5H), 5.08 (s, 2H).

### Preparation 50: 6,7-difluoro-3-(4-methoxyphenyl)-3-morpholinoindolin-2-one (compound 50)

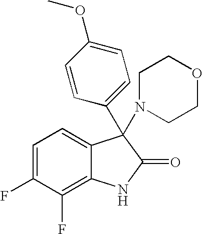

6,7-Difluoro-3-hydroxy-3-(4-methoxyphenyl)indolin-2-one (WO2005097107) (245 mg, 0.84 mmol) was dissolved in DCM (dried), cooled to 0° C., pyridine (82 μl, 1.01 mmol) and thionylchloride (74 μl, 1.01 mmol) were added with stirring. After 2 h, morpholine (73 μl, 1.01 mmol) and DIEA (398 μl, 2.28 mmol) were added, and the mixture allowed to reach rt and stirred overnight. The reaction mixture was extracted with H₂O, brine, dried over Mg₂SO₄, filtered and concentrated. The residue was purified by chromatography (petroleum ether:EtOAc 20:1 to 5:1) to afford compound 50. ¹H-NMR (CDCl₃) δ 8.50 (bs, 1H), 7.38 (m, 2H), 6.96 (m, 1H), 6.79 (m, 3H), 3.71 (s, 3H), 3.62 (m, 4H), 2.52 (m, 4H).

### Preparation 51: 3-hydroxy-3-(thiazol-2-yl)-7-(trifluoromethyl)indolin-2-one (compound 51)

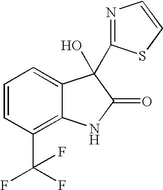

General procedure 1. Starting materials: 7-(trifluoromethyl)indoline-2,3-dione and thiazol-2-yllithium. ¹H-NMR (DMSO-d₆) δ 11.08 (bs, 1H), 7.73 (d, 1H), 7.68 (d, 1H), 7.58 (s, 1H), 7.57 (d, 1H), 7.41 (d, 1H), 7.15 (t, 1H).

### Preparation 52: 7-chloro-3-cyclohexyl-3-hydroxy-6-methylindolin-2-one (compound 52)

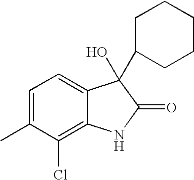

General procedure 1. Starting materials: 7-chloro-6-methylindoline-2,3-dione and cyclohexylmagnesium chloride. ¹H-NMR (DMSO-d₆) δ 10.53 (bs, 1H), 7.09 (d, 1H), 6.94 (d, 1H), 5.84 (s, 1H), 2.20 (s, 3H), 1.9-0.85 (m, 10H), 0.65 (m, 1H).

### Preparation 53: 7-chloro-3-cyclopentyl-3-hydroxy-6-methylindolin-2-one (compound 53)

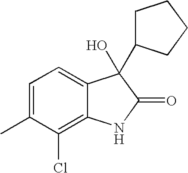

General procedure 1. Starting materials: 7-chloro-6-methylindoline-2,3-dione and cyclopentylmagnesium bromide. ¹H-NMR (DMSO-d₆) δ 10.53 (bs, 1H), 7.15 (d, 1H), 6.94 (d, 1H), 5.90 (s, 1H), 2.30 (s, 3H), 1.75-1.25 (m, 8H), 1.10 (m, 1H).

### Preparation 54: 7-chloro-3-cycloheptyl-3-hydroxy-6-methylindolin-2-one (compound 54)

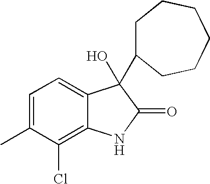

General procedure 1. Starting materials: 7-chloro-6-methylindoline-2,3-dione and cycloheptylmagnesium bromide. ¹H-NMR (DMSO-d₆) δ 10.55 (bs, 1H), 7.10 (d, 1H), 6.93 (d, 1H), 5.91 (s, 1H), 2.30 (s, 3H), 2.06 (m, 1H), 1.91 (m, 1H), 1.71 (m, 1H), 1.6-1.15 (m, 9H), 0.76 (m, 1H).

### Preparation 55: 7-bromo-3-cyclopentyl-3-hydroxy-6-methylindolin-2-one (compound 55)

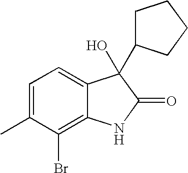

General procedure 1. Starting materials: 7-bromo-6-methylindoline-2,3-dione and cyclopentylmagnesium bromide. ¹H-NMR (CDCl₃) δ 7.79 (bs, 1H), 7.24 (d, 1H), 6.96 (d, 1H), 3.05 (bs, 1H), 2.46 (m, 1H), 2.41 (s, 3H), 1.9-1.4 (m, 7H), 1.27 (m, 1H).

### Preparation 56: 7-bromo-3-cyclohexyl-3-hydroxy-6-methylindolin-2-one (compound 56)

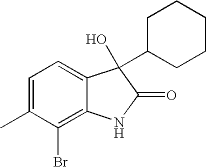

General procedure 1. Starting materials: 7-bromo-6-methylindoline-2,3-dione and cyclohexylmagnesium chloride. ¹H-NMR (DMSO-d₆) δ 10.38 (s, 1H), 7.12 (d, 1H), 6.95 (d, 1H), 5.85 (s, 1H), 2.32 (s, 3H), 1.9-0.9 (m, 10H), 0.66 (m, 1H).

### Preparation 57: 7-bromo-3-cycloheptyl-3-hydroxy-6-methylindolin-2-one (compound 57)

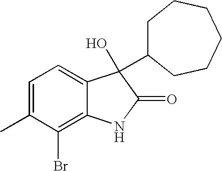

General procedure 1. Starting materials: 7-bromo-6-methylindoline-2,3-dione and cycloheptylmagnesium bromide. ¹H-NMR (DMSO-d₆) δ 10.41 (s, 1H), 7.13 (d, 1H), 6.94 (d, 1H), 5.91 (s, 1H), 2.32 (s, 3H), 2.06 (m, 1H), 1.90 (m, 1H), 1.71 (m, 1H), 1.6-1.15 (m, 9H), 0.76 (m, 1H).

### Preparation 58: 3-cyclopentyl-3-hydroxy-7-methylindolin-2-one (compound 58)

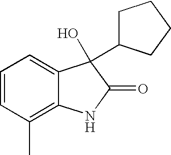

General procedure 1. Starting materials: 7-methylindoline-2,3-dione and cyclopentylmagnesium bromide. MS [M+Na]⁺=254.0, [M−H]⁻=230.0

### Preparation 59: 3-cyclohexyl-3-hydroxy-7-methylindolin-2-one (compound 59)

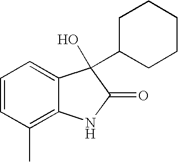

General procedure 1. Starting materials: 7-methylindoline-2,3-dione and cyclohexylmagnesium chloride. ¹H-NMR (DMSO-d₆) δ 10.22 (bs, 1H), 7.03 (m, 2H), 6.86 (t, 1H), 5.66 (s, 1H), 2.19 (m, 1H), 2.17 (s, 3H), 1.9-1.4 (m, 6H), 1.15 (m, 3H), 0.65 (m, 1H).

### Preparation 60: 3-cycloheptyl-3-hydroxy-7-methylindolin-2-one (compound 60)

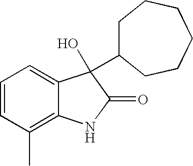

General procedure 1. Starting materials: 7-methylindoline-2,3-dione and cycloheptylmagnesium bromide. ¹H-NMR (DMSO-d₆) δ 10.24 (bs, 1H), 7.04 (m, 2H), 6.85 (t, 1H), 5.73 (s, 1H), 2.17 (s, 3H), 2.07 (m, 1H), 1.80 (m, 1H), 1.72 (m, 1H), 1.6-1.15 (m, 9H), 0.75 (m, 1H).

### Preparation 61: 7-bromo-3-cyclopentyl-3-hydroxyindolin-2-one (compound 61)

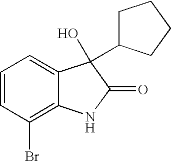

General procedure 1. Starting materials: 7-bromoindoline-2,3-dione and cyclopentylmagnesium bromide. ¹H-NMR (DMSO-d₆) δ 10.49 (bs, 1H), 7.40 (d, 1H), 7.29 (d, 1H), 6.92 (t, 1H), 5.96 (s, 1H), 2.31 (m, 1H), 1.75-1.3 (m, 7H), 1.17 (m, 1H).

### Preparation 63: 7-bromo-3-cyclohexyl-3-hydroxyindolin-2-one (compound 63)

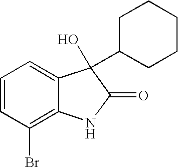

General procedure 1. Starting materials: 7-bromoindoline-2,3-dione and cyclohexylmagnesium chloride. ¹H-NMR (DMSO-d₆) δ 10.49 (bs, 1H), 7.40 (d, 1H), 7.23 (d, 1H), 6.92 (t, 1H), 5.91 (s, 1H), 1.9-0.9 (m, 10H), 0.66 (m, 1H).

### Preparation 63: 7-bromo-3-cycloheptyl-3-hydroxyindolin-2-one (compound 63)

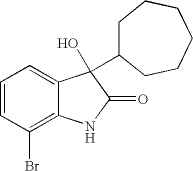

General procedure 1. Starting materials: 7-bromoindoline-2,3-dione and cycloheptylmagnesium bromide. ¹H-NMR (DMSO-d₆) δ 10.52 (bs, 1H), 7.40 (d, 1H), 7.24 (d, 1H), 6.91 (t, 1H), 5.98 (s, 1H), 2.04 (m, 1H), 1.92 (m, 1H), 1.72 (m, 1H), 1.65-1.15 (m, 9H) 0.79 (m, 1H).

### Preparation 64: 3-cyclooctyl-3-hydroxy-7-(trifluoromethyl)indolin-2-one (compound 64)

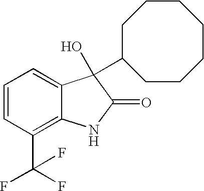

General procedure 1. Starting materials: 7-(trifluoromethyl)indoline-2,3-dione and cyclooctylmagnesium bromide. ¹H-NMR (DMSO-d₆) δ 10.73 (bs, 1H), 7.51 (m, 2H), 7.13 (t, 1H), 6.04 (s, 1H), 2.15-1.2 (m, 14H), 0.86 (m, 1H).

### Preparation 65: 7-chloro-3-cyclooctyl-3-hydroxy-6-methylindolin-2-one (compound 65)

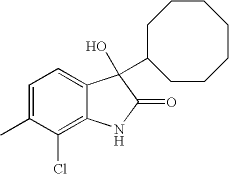

General procedure 1. Starting materials: 7-chloro-6-methylindoline-2,3-dione and cyclooctylmagnesium bromide. ¹H-NMR (DMSO-d₆) δ 10.50 (bs, 1H), 7.06 (d, 1H), 6.84 (d, 1H), 5.84 (s, 1H), 2.23 (s, 3H), 2.15-1.1 (m, 14H), 0.78 (m, 1H).

### Preparation 66: 3-cyclopentyl-3-hydroxy-5,7-dimethylindolin-2-one (compound 66)

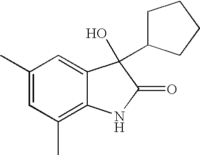

General procedure 1. Starting materials: 5,7-dimethylindoline-2,3-dione and cyclopentylmagnesium bromide. MS [2M+Na]⁺=513.3, [M−H]⁻=244.1

### Preparation 67: 3-cyclohexyl-3-hydroxy-5,7-dimethylindolin-2-one (compound 67)

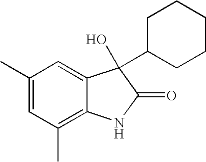

General procedure 1. Starting materials: 5,7-dimethylindoline-2,3-dione and cyclohexylmagnesium chloride. ¹H-NMR (DMSO-d₆) δ 10.11 (bs, 1H), 6.86 (s, 1H), 6.82 (s, 1H), 5.60 (s, 1H), 2.22 (s, 3H), 2.13 (s, 3H), 1.9-0.85 (m, 10H), 0.66 (m, 1H).

### Preparation 68: 3-cycloheptyl-3-hydroxy-5,7-dimethylindolin-2-one (compound 68)

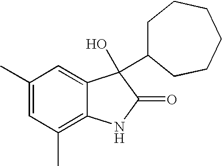

General procedure 1. Starting materials: 5,7-dimethylindoline-2,3-dione and cycloheptylmagnesium bromide. ¹H-NMR (DMSO-d₆) δ 10.14 (bs, 1H), 6.87 (s, 1H), 6.82 (s, 1H), 5.67 (s, 1H), 2.21 (s, 3H), 2.13 (s, 3H), 2.06 (m, 1H), 1.88 (m, 1H), 1.72 (m, 1H), 1.6-1.2 (m, 9H), 0.78 (m, 1H).

### Preparation 69: 3-cyclopentyl-3-hydroxy-5-methoxy-7-methylindolin-2-one (compound 69)

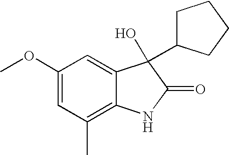

General procedure 1. Starting materials: 5-methoxy-7-methylindoline-2,3-dione and cyclopentylmagnesium bromide. ¹H-NMR (DMSO-d₆) δ 10.07 (bs, 1H), 6.70 (d, 1H), 6.62 (d, 1H), 5.71 (s, 1H), 3.69 (s, 3H), 2.29 (m, 1H), 2.16 (s, 3H), 1.65-1.3 (m, 7H), 1.21 (m, 1H).

### Preparation 70: 3-cyclohexyl-3-hydroxy-5-methoxy-7-methylindolin-2-one (compound 70)

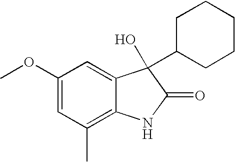

General procedure 1. Starting materials: 5-methoxy-7-methylindoline-2,3-dione and cyclohexylmagnesium chloride. ¹H-NMR (DMSO-d₆) δ 10.07 (bs, 1H), 6.63 (d, 1H), 6.61 (d, 1H), 5.66 (s, 1H), 3.69 (s, 3H), 2.15 (s, 3H), 1.75 (m, 3H), 1.55 (m, 3H), 1.05 (m, 4H), 0.70 (m, 1H).

### Preparation 71: 3-cycloheptyl-3-hydroxy-5-methoxy-7-methylindolin-2-one (compound 71)

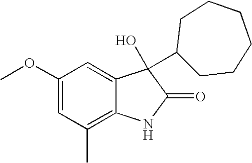

General procedure 1. Starting materials: 5-methoxy-7-methylindoline-2,3-dione and cycloheptylmagnesium bromide. ¹H-NMR (DMSO-d₆) δ 10.09 (bs, 1H), 6.64 (d, 1H), 6.61 (d, 1H), 5.73 (s, 1H), 3.68 (s, 3H), 2.16 (s, 3H), 2.02 (m, 1H), 1.89 (m, 1H), 1.71 (m, 1H), 1.6-1.15 (m, 9H), 0.80 (m, 1H).

### Preparation 72: 5-chloro-3-cyclopentyl-3-hydroxy-7-methylindolin-2-one (compound 72)

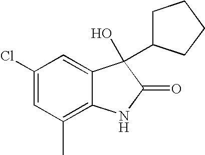

General procedure 1. Starting materials: 5-chloro-7-methylindoline-2,3-dione and cyclopentylmagnesium bromide. ¹H-NMR (DMSO-d₆) δ 10.40 (bs, 1H), 7.11 (m, 2H), 5.90 (s, 1H), 2.31 (m, 1H), 2.18 (s, 3H), 1.9-1.3 (m, 7H), 1.21 (m, 1H).

### Preparation 73: 5-chloro-3-cyclohexyl-3-hydroxy-7-methylindolin-2-one (compound 73)

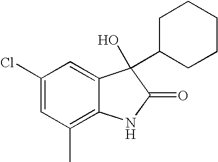

General procedure 1. Starting materials: 5-chloro-7-methylindoline-2,3-dione and cyclohexylmagnesium chloride. ¹H-NMR (DMSO-d₆) δ 10.40 (bs, 1H), 7.11 (d, 1H), 7.04 (d, 1H), 5.85 (s, 1H), 2.17 (s, 3H), 1.9-1.4 (m, 7H), 1.2-0.9 (m, 3H), 0.70 (m, 1H).

### Preparation 74: 3-chloro-3-cycloheptyl-3-hydroxy-7-methylindolin-2-one (compound 74)

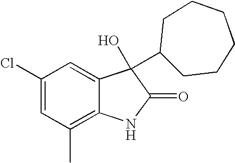

General procedure 1. Starting materials: 5-methoxy-7-methylindoline-2,3-dione and cycloheptylmagnesium bromide. MS [M+Na]⁺=316.1, [M−H]⁻=292.2

### Preparation 75: 3-cyclopentyl-5-fluoro-3-hydroxy-7-methylindolin-2-one (compound 75)

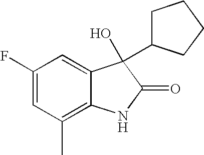

General procedure 1. Starting materials: 5-fluoro-7-methylindoline-2,3-dione and cyclopentylmagnesium bromide. MS [M+Na]⁺=272.1, [M−H]⁻=248.1

### Preparation 76: 3-cyclohexyl-5-fluoro-3-hydroxy-7-methylindolin-2-one (compound 76)

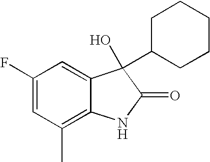

General procedure 1. Starting materials: 5-fluoro-7-methylindoline-2,3-dione and cyclohexylmagnesium chloride. ¹H-NMR (DMSO-d₆) δ 10.28 (bs, 1H), 6.87 (m, 2H), 5.85 (s, 1H), 2.18 (s, 3H), 1.85-1.45 (m, 6H), 1.2-0.9 (m, 4H), 0.69 (m, 1H).

### Preparation 77: 3-cycloheptyl-5-fluoro-3-hydroxy-7-methylindolin-2-one (compound 77)

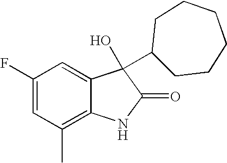

General procedure 1. Starting materials: 5-fluoro-7-methylindoline-2,3-dione and cycloheptylmagnesium bromide. MS [M+Na]⁺=290.1, [M−H]⁻=276.1

### Preparation 78: 3-cyclopentyl-6-fluoro-3-hydroxy-5,7-dimethylindolin-2-one (compound 78)

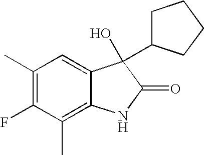

General procedure 1. Starting materials: 6-fluoro-5,7-dimethylindoline-2,3-dione and cyclopentylmagnesium bromide. MS [M+H]⁺=264.1, [M−H]⁻=262.1

### Preparation 79: 3-cyclohexyl-6-fluoro-3-hydroxy-5,7-dimethylindolin-2-one (compound 79)

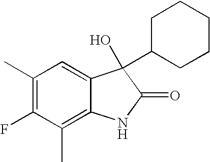

General procedure 1. Starting materials: 6-fluoro-5,7-dimethylindoline-2,3-dione and cyclohexylmagnesium chloride. ¹H-NMR (DMSO-d₆) δ 10.33 (bs, 1H), 6.95 (d, 1H), 5.63 (s, 1H), 2.17 (d, 3H), 2.09 (d, 3H), 1.9-1.4 (m, 6H), 1.2-0.9 (m, 4H), 0.66 (m, 1H).

### Preparation 80: 3-cycloheptyl-6-fluoro-3-hydroxy-5,7-dimethylindolin-2-one (compound 80)

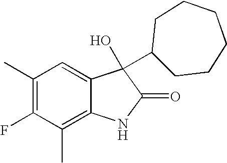

General procedure 1. Starting materials: 6-fluoro-5,7-dimethylindoline-2,3-dione and cycloheptylmagnesium bromide. ¹H-NMR (DMSO-d₆) δ 10.35 (bs, 1H), 6.95 (d, 1H), 5.73 (s, 1H), 2.15 (d, 3H), 2.09 (d, 3H), 2.10 (m, 1H), 1.88 (m, 1H), 1.74 (m, 1H), 1.65-1.0 (m, 9H), 0.78 (m, 1H).

### Preparation 81: 3-cyclopentyl-3-hydroxy-7-methyl-6-(trifluoromethyl)indolin-2-one (compound 81)

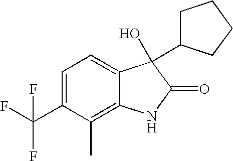

General procedure 1. Starting materials: 7-methyl-6-(trifluoromethyl)indoline-2,3-dione and cyclopentylmagnesium bromide. MS [M+Na]⁺=322.2, [M−H]=298.0.

### Preparation 82: 3-cyclohexyl-3-hydroxy-7-methyl-6-(trifluoromethyl)indolin-2-one (compound 82)

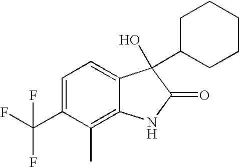

General procedure 1. Starting materials: 7-methyl-6-(trifluoromethyl)indoline-2,3-dione and cyclohexylmagnesium chloride. MS [M+Na]⁺=336.2, [M−H]⁻=312.0.

### Preparation 83: 3-cycloheptyl-3-hydroxy-7-methyl-6-(trifluoromethyl)indolin-2-one (compound 83)

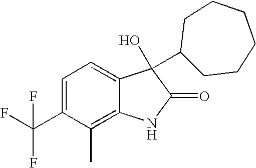

General procedure 1. Starting materials: 7-methyl-6-(trifluoromethyl)indoline-2,3-dione and cycloheptylmagnesium bromide. MS [M+Na]⁺=350.2, [M−H]⁻=326.2.

### Preparation 84: 3-cyclopentyl-3-hydroxy-5-methoxy-6,7-dimethylindolin-2-one (compound 84)

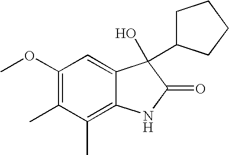

General procedure 1. Starting materials: 5-methoxy-6,7-dimethylindoline-2,3-dione and cyclopentylmagnesium bromide. MS [2M+H]⁺=572.9, [M−H]⁻=274.0.

### Preparation 85: 3-cyclohexyl-3-hydroxy-5-methoxy-6,7-dimethylindolin-2-one (compound 85)

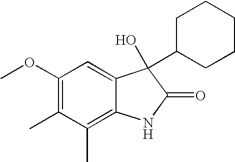

General procedure 1. Starting materials: 5-methoxy-6,7-dimethylindoline-2,3-dione and cyclohexylmagnesium chloride. MS [2M+Na]⁺=600.9, [M−H]⁻=288.1.

### Preparation 83: 3-cycloheptyl-3-hydroxy-5-methoxy-6,7-dimethylindolin-2-one (compound 83)

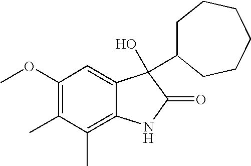

General procedure 1. Starting materials: 5-methoxy-6,7-dimethylindoline-2,3-dione and cycloheptylmagnesium bromide. MS [M+H]⁺=304.2, [M−H]⁻=302.2.

### EXAMPLES

### Example 1

### 3-ethynyl-6,7-difluoro-3-(4-hydroxyphenyl)indolin-2-one (compound 1001)

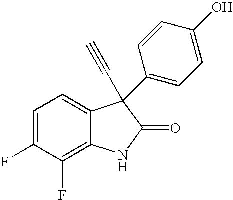

General procedure 2. Starting materials: compound 1. ¹H-NMR (DMSO-d₆) δ 11.55 (bs, 1H), 9.57 (s, 1H), 7.12-6.9 (m, 4H), 6.75 (d, 2H), 3.62 (s, 1H).

### Example 2

### 3-benzyl-6,7-difluoro-3-(4-hydroxyphenyl)indoline-2-one (compound 1002)

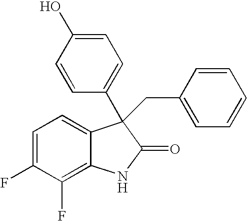

General procedure 5. Starting materials: compound 4. ¹H-NMR (DMSO-d₆) δ 11.05 (bs, 1H), 9.51 (s, 1H), 7.3-7.2 (m, 3H), 7.18-7.11 (m, 3H), 7.08 (m, 1H), 6.8 (d, 2H), 3.56 (dd, 2H).

### Example 3

### 6,7-difluoro-3-(4-hydroxyphenyl)-3-methylindolin-2-one (compound 1003)

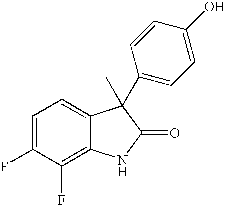

General procedure 2. Starting materials: compound 5. ¹H-NMR (DMSO-d₆) δ 11.20 (bs, 1H), 9.40 (bs, 1H), 7.05-6.95 (m, 4H), 6.69 (d, 2H), 1.62 (s, 3H).

### Example 4

### 3-cyclopentyl-6,7-difluoro-3-(4-hydroxyphenyl)indolin-2-one (compound 1004)

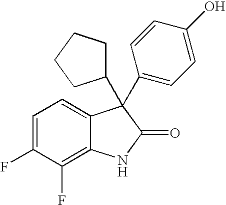

General procedure 2. Starting materials: compound 6. ¹H-NMR (DMSO-d₆) δ 11.21 (bs, 1H), 9.38 (bs, 1H), 7.16-7.08 (m, 4H), 7.01 (m, 1H), 6.69 (d, 2H), 2.97 (m, 1H), 1.6-1.2 (m, 7H), 0.94 (m, 1H).

### Example 5

### 3-(cyclohexylmethyl)-6,7-difluoro-3-(4-hydroxyphenyl)indolin-2-one (compound 1005)

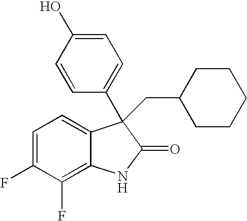

General procedure 2. Starting materials: compound 7. ¹H-NMR (DMSO-d₆) δ 11.19 (bs, 1H), 9.38 (bs, 1H), 7.12-6.9 (m, 4H), 6.67 (d, 2H), 2.12 (m, 2H), 1.6-1.4 (m, 7H), 1.25 (m, 1H), 1.1-0.7 (m, 6H).

### Example 6

### 6,7-difluoro-3-(4-hydroxyphenyl)-3-(pyridin-4-yl)indolin-2-one (compound 1006)

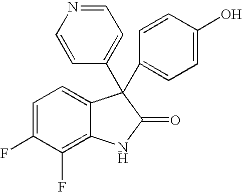

General procedure 2. Starting material: compound 8. ¹H-NMR (DMSO-d₆) δ 10.82 (bs, 1H), 9.01 (bs, 3H), 7.70 (bs, 2H), 7.68-7.42 (m, 4H), 7.29 (m, 2H).

### Example 7

### 6,7-difluoro-3-(4-hydroxyphenyl)-3-isopropylindolin-2-one (compound 1007)

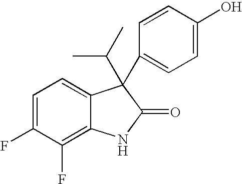

General procedure 2. Starting material: compound 9. ¹H-NMR (CDCl₃) δ 7.87 (bs, 1H), 7.16 (d, 2H), 6.97 (m, 1H), 6.84 (m, 1H), 6.66 (2, 2H), 3.1 (bs, 1H), 2.77 (m, 1H) 0.89 (d, 3H), 0.68 (d, 3H).

### Example 8

### 6,7-difluoro-3-(4-hydroxyphenyl)-3-(thiophen-2-yl)indolin-2-one (compound 1008)

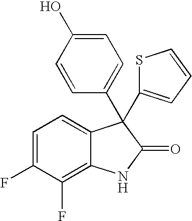

General procedure 2. Starting material: compound 10. ¹H-NMR (DMSO-d₆) δ 11.54 (bs, 1H), 9.52 (bs, 1H), 7.51 (d, 1H), 7.2-6.9 (m, 6H), 6.70 (d, 2H).

### Example 9

### 3-butyl-6,7-difluoro-3-(4-hydroxyphenyl)indolin-2-one (compound 1009)

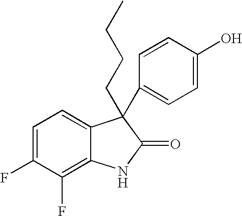

General procedure 2. Starting material: compound 11. ¹H-NMR (DMSO-d₆) δ 11.21 (bs, 1H), 9.39 (bs, 1H), 7.1-6.9 (m, 4H), 6.68 (d, 2H), 2.13 (m, 2H), 1.21 (m, 2H), 0.98 (m, 1H), 0.78 (m, 4H).

### Example 10

### 3-cyclohexyl-6,7-difluoro-3-(4-hydroxyphenyl)indolin-2-one (compound 1010)

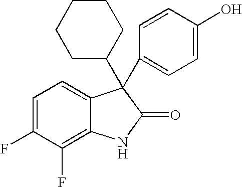

General procedure 2. Starting material: compound 12. ¹H-NMR (CDCl₃) δ 8.46 (bs, 1H), 7.21 (d, 2H), 7.05 (m, 1H), 6.92 (m, 1H), 6.75 (d, 2H), 6.13 (bs, 1H), 2.48 (m, 1H) 1.8-1.0 (m, 9H), 0.69 (m, 1H).

### Example 11

### 6,7-difluoro-3-(4-hydroxyphenyl)-3-propylindolin-2-one (compound 1011)

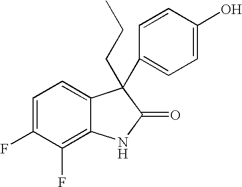

General procedure 2. Starting material: compound 13. ¹H-NMR (DMSO-d₆) δ 11.20 (bs, 1H), 9.38 (bs, 1H), 7.12-7.0 (m, 4H), 6.68 (d, 2H), 2.13 (m, 2H), 1.21 (m, 2H), 1.02 (m, 1H), 0.83 (m, 4H).

### Example 12

### 6,7-difluoro-3-(4-hydroxyphenyl)-3-pentylindolin-2-one (compound 1012)

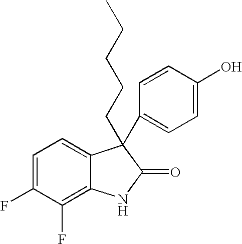

General procedure 2. Starting material: compound 14. ¹H-NMR (DMSO-d₆) δ 11.21 (bs, 1H), 9.39 (bs, 1H), 7.1-6.9 (m, 4H), 6.68 (d, 2H), 2.10 (m, 2H), 1.26-0.94 (m, 5H), 0.78 (m, 4H).

### Example 13

### 6,7-difluoro-3-(4-hydroxyphenyl)-3-pentylindolin-2-one (compound 1013)

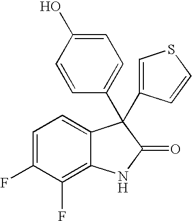

General procedure 2. Starting material: compound 15. ¹H-NMR (DMSO-d₆) δ 11.20 (bs, 1H), 9.43 (bs, 1H), 7.52 (d, 1H), 7.15 (m, 2H), 7.05 (m, 2H), 6.88 (d, 2H), 6.67 (d, 2H).

### Example 14

### 6,7-difluoro-3-(4-hydroxyphenyl)-3-(pyridin-3-yl)indolin-2-one (compound 1014)

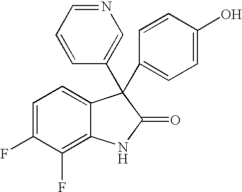

General procedure 2. Starting material: compound 16. MS [M+K]⁺=380.20.

### Example 15

### 6,7-difluoro-3-(4-hydroxyphenyl)-3-(pyridin-4-yl N-oxide)indolin-2-one (compound 1015)

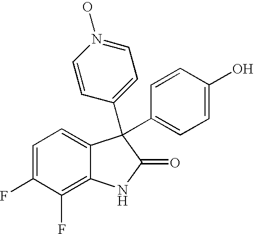

Starting material: example 6. Compound 1006 (26 mg, 0.077 mmol) was dissolved in DCM, and peracetic acid in acetic acid (39%, 0.125 mL) was added and the mixture stirred overnight, concentrated and purified by flash chromatography (chloroform:methanol: 25% ammonia 95:5:0.5) to yield compound 1015 (19 mg). ¹H-NMR (DMSO-d₆) δ 11.67 (bs, 1H), 9.60 (bs, 1H), 8.18 (d, 2H), 7.25-7.00 (m, 4H), 6.97 (d, 2H), 6.74 (d, 2H). MS [M+H]⁺=355.20.

### Example 16

### 3-(but-3-en-2-yl)-6,7-difluoro-3-(4-hydroxyphenyl)indolin-2-one (2 diastereoisomers) (compound 1016)

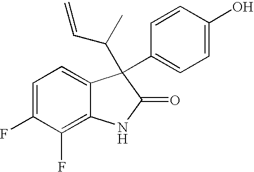

General procedure 2. Starting material: compound 17. ¹H-NMR (DMSO-d₆) δ 11.23+11.16 (s, 1H), 9.40 (s, 1H), 7.18-7.0 (m, 4H), 6.71 (d, 2H), 5.73+5.30 (m, 1H), 4.95 (m, 2H), 3.33 (m, 1H), 0.94+0.76 (d, 3H).

### Example 17

### 3-sec-butyl-6,7-difluoro-3-(4-hydroxyphenyl)indolin-2-one (2 diastereoisomers) (compound 1017)

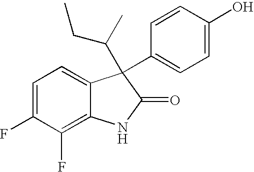

General procedure 2. Starting material: compound 18. ¹H-NMR (CDCl₃) δ 7.62, 7.14 (m, 3H), 6.95 (m, 1H), 6.83 (m, 1H), 6.69 (d, 2H), 5.0 (s, 1H), 2.45 (m, 1H), 1.5-0.98 (m, 2H), 0.86+0.76 (d, 3H), 0.80 (m, 3H).

### Example 18

### 3-cycloheptyl-6,7-difluoro-3-(4-hydroxyphenyl)indolin-2-one (compound 1018)

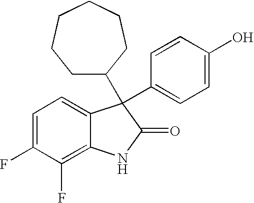

General procedure 2. Starting material: compound 19. ¹H-NMR (CDCl₃) δ 7.51 (bs, 1H), 7.14 (d, 2H), 6.96 (m, 1H), 6.82 (m, 2H), 6.68 (d, 2H), 4.89 (s, 1H) 2.57 (m, 1H), 1.7-1.1 (m, 11H), 0.81 (m, 1H).

### Example 19

### 3-(1-(benzyloxy)-1H-pyrazol-4-yl)-6,7-difluoro-3-(4-hydroxyphenyl)indolin-2-one (compound 1019)

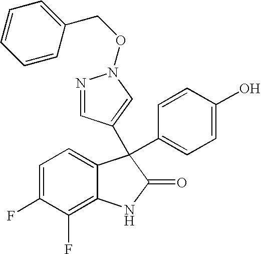

General procedure 2. Starting material: compound 20. ¹H-NMR (CDCl₃) δ 7.96 (bs, 1H), 7.35-7.15 (m, 5H), 7.09 (s, 1H), 6.87 (s, 1H), 6.85-6.65 (m, 4H), 6.60 (d, 2H), 5.23 (s, 2H), 5.19 (s, 1H). Example 20: 6,7-difluoro-3-(1-hydroxy-1H-pyrazol-4-yl)-3-(4-hydroxyphenyl)indolin-2-one (compound 1020)

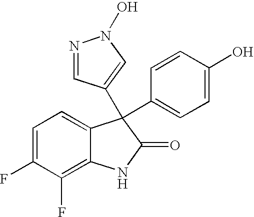

Starting material: Example 19. Compound 1019 (40 mg, 0.09 mmol) was dissolved in methanol (4 mL) and the solution bubbled through with N₂ for 2 minutes. 10% Pd/C (3.1 mg) was added. The flask was fitted with a septum and a N₂-filled balloon, carefully evacuated and filled with N₂. The N₂-filled balloon was substituted with a H₂-filled balloon, the flask was then carefully evacuated and filled with H₂ twice and the reaction mixture vigorously stirred at 0° C. for 30 minutes. The flask was the carefully evacuated and filled with N₂ twice, the reaction mixture filtered through Celite, concentrated and purified by flash chromatography (chloroform:methanol: 25% ammonia 80:20:1) to yield Example 20 (26 mg). ¹H-NMR (DMSO-d₆) δ 7.33 (s, 1H), 7.14 (m, 1H), 7.01 (m, 1H), 6.95 (s, 1H), 6.87 (d, 2H), 6.66 (d, 2H), 4-3 (bs, >3H). MS [M+H]⁺=344.15.

### Example 21

### 3-cyclohexyl-3-(4-hydroxyphenyl)-7-(trifluoromethyl)indolin-2-one (compound 1021)

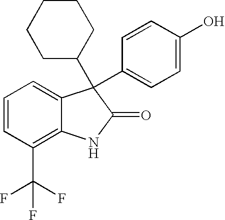

General procedure 2. Starting material: compound 21. ¹H-NMR (CDCl₃) δ 8.02 (bs, 1H), 7.52 (m, 2H), 7.23 (m, 3H), 6.87 (s, 1H), 6.76 (d, 2H), 5.91 (bs, 1H), 2.52 (m, 1H), 2.15-0.95 (m, 9H), 0.70 (m, 1H).

### Example 22

### 3-(3,4-difluorophenyl)-6,7-difluoro-3-(4-hydroxyphenyl)indolin-2-one (compound 1022)

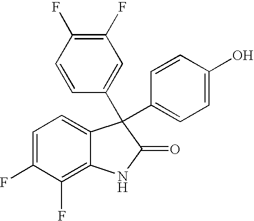

General procedure 2. Starting material: compound 22. ¹H-NMR (CDCl₃) δ 7.96 (bs, 1H), 7.1-6.76 (m, 7H), 6.69 (d, 2H), 5.05 (s, 1H).

### Example 23

### 6,7-difluoro-3-(3-fluor-4-methylphenyl)-3-(4-hydroxyphenyl)indolin-2-one (compound 1023)

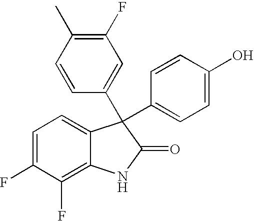

General procedure 2. Starting material: compound 23. ¹H-NMR (DMSO-d₆) δ 11.48 (bs, 1H), 9.49 (s, 1H), 7.24 (m, 1H), 7.14-6.8 (m, 6H), 6.72 (d, 2H), 2.20 (s, 3H).

### Example 24

### 6-chloro-3-cyclohexyl-3-(4-hydroxyphenyl)-7-methylindolin-2-one (compound 1024)

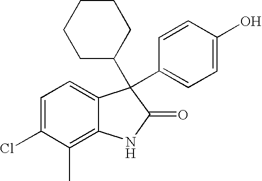

General procedure 2. Starting material: compound 24. ¹H-NMR (DMSO-d₆) δ 10.67 (bs, 1H), 9.35 (s, 1H), 7.12 (m, 4H), 6.69 (d, 2H), 2.32 (m, 1H), 2.25 (s, 3H), 1.59 (m, 3H), 1.40 (m, 2H), 1.13 (m, 3H), 0.96 (m, 1H), 0.73 (m, 1H).

### Example 25

### 3-cyclohexyl-3-(4-hydroxyphenyl)-6,7-dimethylindolin-2-one (compound 1025)

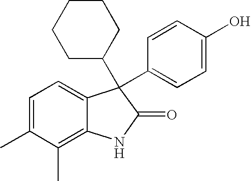

General procedure 2. Starting material: compound 25. ¹H-NMR (DMSO-d₆) δ 10.35 (bs, 1H), 9.29 (s, 1H), 7.13 (m, 2H), 7.01 (d, 1H), 6.85 (d, 1H), 6.68 (d, 2H), 2.25 (m, 1H), 2.23 (s, 3H), 2.12 (s, 3H), 1.75-0.8 (m, 9H), 0.67 (m, 1H).

### Example 26

### 3-(cyclopentylmethyl)-6,7-difluoro-3-(4-hydroxyphenyl)indolin-2-one (compound 1026)

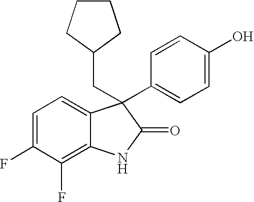

General procedure 2. Starting material: compound 26. ¹H-NMR (DMSO-d₆) δ 11.21 (bs, 1H), 9.39 (s, 1H), 7.05 (m, 4H), 6.68 (d, 2H), 2.29 (m, 2H), 1.6-1.0 (m, 8H), 0.86 (m, 1H).

### Example 27

### 3-cyclohexyl-6,7-difluoro-3-(4-hydroxyphenyl)indolin-2-one (enantiomer 1) (compound 1027)

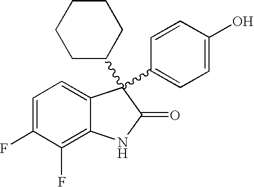

General procedure 6 (heptane:ethanol 70:30, 7 mL/min). Starting material: compound 1010. t_{R} (Chiralcel OD 250×4.6 mm ID 5 micron, heptane:ethanol 70:30, 0.6 mL/min): 8.9 min.

### Example 28

### 3-cyclohexyl-6,7-difluoro-3-(4-hydroxyphenyl)indolin-2-one (enantiomer 2) (compound 1028)

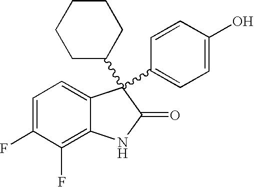

General procedure 6. Starting material: Compound 1010. t_{R} (Chiralcel OD 250×4.6 mm ID 5 micron, heptane:ethanol 70:30, 0.6 mL/min): 14.5 min.

### Example 29

### 6,7-difluoro-3-(4-hydroxy-3-methylphenyl)-3-(4-hydroxyphenyl)indolin-2-one (compound 1029)

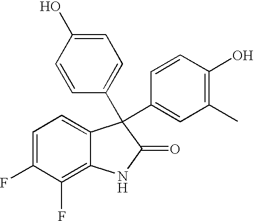

General procedure 5. Starting material: compound 27. ¹H-NMR (DMSO-d₆) δ 11.34 (bs, 1H), 9.29 (bd, 2H), 7.05-6.9 (m, 4H), 6.83 (d, 1H), 6.8-6.6 (m, 4H), 2.03 (s, 3H).

### Example 30

### 6,7-difluoro-3-(4-hydroxy-2-methylphenyl)-3-(4-hydroxyphenyl)indolin-2-one (compound 1030)

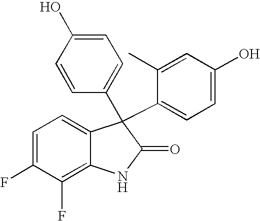

General procedure 5. Starting material: compound 28.

¹H-NMR (DMSO-d₆) δ 10.98 (bs, 1H), 9.46 (bs, 1H), 9.40 (bs), 7.11 (d, 2H), 6.92 (m, 1H), 6.73 (m, 3H), 6.52 (m, 3H), 2.17 (s, 3H).

### Example 31

### 3-cyclooctyl-6,7-difluoro-3-(4-hydroxyphenyl)indolin-2-one (compound 1031)

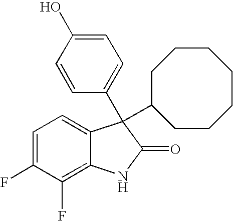

General procedure 2. Starting material: compound 29. ¹H-NMR (DMSO-d₆) δ 11.16 (bs, 1H), 9.40 (bs, 1H), 7.20-6.90 (m, 4H), 7.05 (d, 2H), 2.65 (m, 1H), 1.7-1.2 (m, 13H), 0.89 (m, 1H).

### Example 32

### 6,7-difluoro-3-(4-hydroxyphenyl)-3-(naphthalene-1-yl)indolin-2-one (compound 1032)

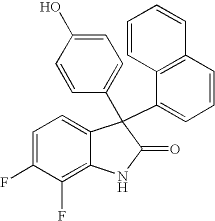

General procedure 2. Starting material: compound 30. ¹H-NMR (DMSO-d₆) δ 11.55 (bs, 1H), 9.53 (bs, 1H), 7.92 (t, 2H), 7.6-7.2 (m, 4H), 7.2-6.8 (m, 5H), 6.72 (d, 2H).

### Example 33

### 6,7-difluoro-3-(4-hydroxyphenyl)-3-(naphthalene-2-yl)indolin-2-one (compound 1033)

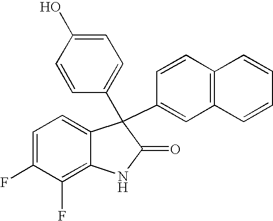

General procedure 2. Starting material: compound 31. ¹H-NMR (DMSO-d₆) δ 11.55 (bs, 1H), 9.53 (bs, 1H), 7.92-7.80 (m, 3H), 7.62 (s, 1H), 7.50 (m, 2H), 7.34 (dd, 1H), 7.15 (m, 1H), 7.06 (m, 1H), 7.00 (d, 2H), 6.74 (d, 2H).

### Example 34

### 3-cycloheptyl-7-fluoro-3-(4-hydroxyphenyl)-6-methylindolin-2-one (compound 1034)

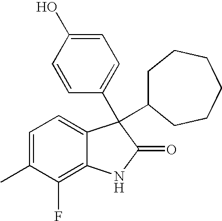

General procedure 2. Starting material: compound 32. ¹H-NMR (CDCl₃) δ 7.64 (bs, 1H), 7.13 (d, 2H), 6.92 (d, 1H), 6.82 (t, 1H), 6.64 (d, 2H), 5.51 (bs, 1H), 2.56 (m, 1H), 2.26 (d, 3H), 1.7-1.3 (m, 10H), 0.80 (m, 2H).

### Example 35

### 3-cyclohexyl-7-fluoro-3-(4-hydroxyphenyl)-6-methylindolin-2-one (compound 1035)

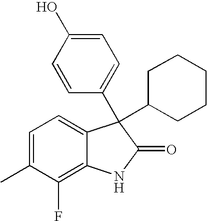

General procedure 2. Starting material: compound 33. ¹H-NMR (DMSO-d₆) δ 10.82 (bs, 1H), 9.33 (bs, 1H), 7.12 (m, 2H), 7.04 (d, 1H), 6.92 (t, 1H), 6.70 (m, 2H), 2.30 (m, 4H), 1.7-0.8 (m, 9H), 0.68 (m, 1H).

### Example 36

### 3-tert-butyl-6,7-difluoro-3-(4-hydroxyphenyl)indolin-2-one (compound 1036)

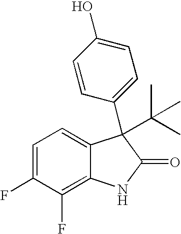

General procedure 2. Starting material: compound 34. ¹H-NMR (DMSO-d₆) δ 11.32 (bs, 1H), 9.41 (bs, 1H), 7.63 (m, 3H), 7.03 (m, 1H), 6.71 (m, 2H), 0.95 (s, 3H).

### Example 37

### 6,7-difluoro-3-(4-hydroxyphenyl)-3-tert-pentylindolin-2-one (compound 1037)

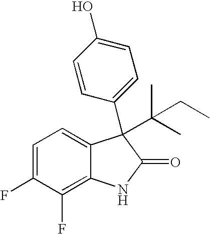

General procedure 2. Starting material: compound 35. ¹H-NMR (DMSO-d₆) δ 11.32 (bs, 1H), 9.41 (bs, 1H), 7.63 (m, 3H), 7.01 (m, 1H), 6.71 (m, 2H), 1.3 (m, 2H), 0.94 (d, 6H), 0.69 (t, 3H).

### Example 38

### 3-cyclopentyl-6-fluoro-3-(4-hydroxyphenyl)-7-methylindolin-2-one (compound 1038)

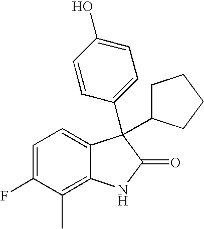

General procedure 2. Starting material: compound 36. ¹H-NMR (CDCl₃) δ 8.5 (bs, 1H), 7.16 (m, 2H), 7.00 (m, 1H), 6.6 (m, 3H), 5.5 (bs, 1H), 2.96 (m, 1H), 2.12 (s, 3H), 1.7-0.65 (m, 9H).

### Example 39

### 3-cyclohexyl-6-fluoro-3-(4-hydroxyphenyl)-7-methylindolin-2-one (compound 1039)

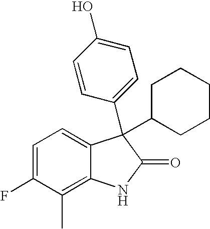

General procedure 2. Starting material: compound 37. ¹H-NMR (DMSO-d₆) δ 10.65 (bs, 1H), 9.33 (bs, 1H), 7.13 (m, 3H), 6.80 (m, 1H), 6.69 (m, 2H), 2.31 (m, 1H), 2.15 (s, 3H), 1.7-0.8 (m, 9H), 0.70 (m, 1H).

### Example 40

### 6-fluoro-3-(4-hydroxyphenyl)-7-methyl-3-pentylindolin-2-one (compound 1040)

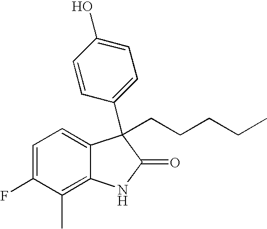

General procedure 2. Starting material: compound 38. ¹H-NMR (CDCl₃) δ 10.68 (bs, 1H), 9.34 (bs, 1H), 7.06 (m, 2H), 7.02 (m, 1H), 6.77 (m, 1H), 6.67 (m, 2H), 2.17 (s, 3H), 2.08 (m, 2H), 1.19 (m, 4H), 1.00 (m, 1H), 0.9-0.7 (m, 4H).

### Example 41

### 3-cycloheptyl-6-fluoro-3-(4-hydroxyphenyl)-7-methylindolin-2-one (compound 1041)

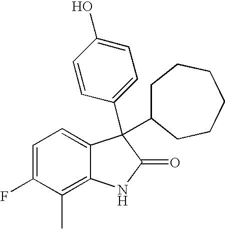

General procedure 2. Starting material: compound 39. ¹H-NMR (DMSO-d₆) δ 10.63 (bs, 1H), 9.35 (bs, 1H), 7.1 (m, 3H), 6.80 (m, 1H), 6.70 (m, 2H), 2.47 (m, 1H), 2.14 (s, 3H), 1.7-1.1 (m, 11H), 0.85 (m, 1H).

### Example 42

### 3-cycloheptyl-3-(4-hydroxyphenyl)-7-(trifluoromethyl)indolin-2-one (compound 1042)

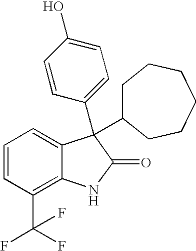

General procedure 2. Starting material: compound 40. ¹H-NMR (DMSO-d₆) δ 10.90 (bs, 1H), 9.42 (bs, 1H), 7.56 (m, 2H), 7.21 (m, 1H), 7.09 (m, 2H), 6.71 (m, 2H), 2.55 (m, 1H), 1.7-1.2 (m, 11H), 0.86 (m, 1H).

### Example 43

### 3-cycloheptyl-3-(4-hydroxyphenyl)-6,7-dimethylindolin-2-one (compound 1043)

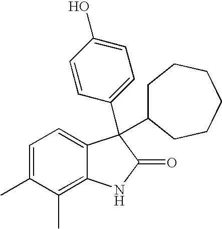

General procedure 2. Starting material: compound 41. ¹H-NMR (DMSO-d₆) δ 10.31 (bs, 1H), 9.29 (bs, 1H), 7.10 (m, 2H), 6.99 (d, 1H), 6.84 (d, 1H), 6.67 (m, 2H), 2.46 (m, 1H), 2.23 (s, 3H), 2.12 (s, 3H), 1.65-1.2 (m, 11H), 0.85 (m, 1H).

### Example 44

### 6-chloro-3-cycloheptyl-3-(4-hydroxyphenyl)-7-methylindolin-2-one (compound 1044)

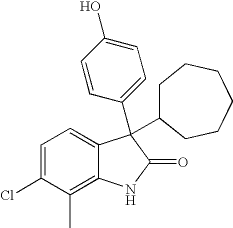

General procedure 2. Starting material: compound 42. ¹H-NMR (DMSO-d₆) δ 10.63 (bs, 1H), 9.36 (bs, 1H), 7.09 (m, 4H), 6.69 (m, 2H), 2.26 (s, 3H), 1.7-1.15 (m, 12H), 0.85 (m, 1H).

### Example 45

### 3-cyclopentyl-3-(4-hydroxyphenyl)-6-methoxy-7-methylindolin-2-one (compound 1045)

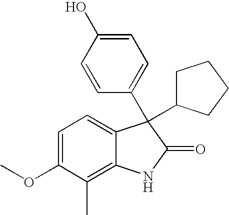

General procedure 2. Starting material: compound 43. ¹H-NMR (DMSO-d₆) δ 10.40 (bs, 1H), 9.27 (bs, 1H), 7.13 (m, 2H), 7.05 (d, 1H), 6.67 (m, 2H), 6.58 (d, 1H), 3.78 (s, 3H), 2.93 (m, 1H), 2.07 (s, 3H), 1.6-1.2 (m, 7H), 0.86 (m, 1H).

### Example 46

### 3-cyclohexyl-3-(4-hydroxyphenyl)-6-methoxy-7-methylindolin-2-one (compound 1046)

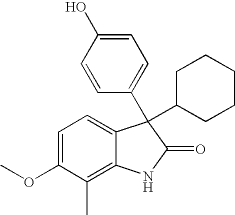

General procedure 2. Starting material: compound 44. ¹H-NMR (DMSO-d₆) δ 10.39 (bs, 1H), 9.28 (bs, 1H), 7.14 (m, 2H), 7.07 (d, 1H), 6.68 (m, 2H), 6.61 (d, 1H), 3.79 (s, 3H), 2.28 (m, 1H), 2.05 (s, 3H), 1.55 (m, 3H), 1.4 (m, 2H), 1.16 (m, 3H), 0.96 (m, 1H), 0.69 (m, 1H).

### Example 47

### 3-(4-hydroxyphenyl)-3-(1H-imidazol-1-yl)-7-(trifluoromethyl)indolin-2-one (compound 1047)

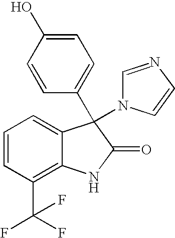

Compound 47 (184 mg, 0.36 mmol) was dissolved in methanol (10 mL) and the solution bubbled through with N₂ for 2 minutes. 10% Pd/C (60 mg) was added. The flask was fitted with a septum and a N₂-filled balloon, carefully evacuated and filled with N₂. The N₂-filled balloon was substituted with a H₂-filled balloon, the flask was then carefully evacuated and filled with H₂ twice and the reaction mixture vigorously stirred at rt for 4 h. The flask was the carefully evacuated and filled with N₂ twice, the reaction mixture filtered through Celite, concentrated and purified by flash chromatography (1%-5% methanol in DCM) to yield compound 1047. ¹H-NMR (DMSO-d₆) δ 11.53 (bs, 1H), 9.79 (bs, 1H), 7.78 (d, 1H), 7.68 (d, 1H), 7.56 (s, 1H), 7.28 (t, 1H), 7.1-6.9 (m, 4H), 6.80 (d, 2H).

### Example 48

### 6,7-difluoro-3-(4-hydroxyphenyl)-3-(1H-imidazol-1-yl)indolin-2-one (compound 1048)

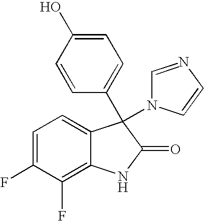

Compound 49 (127 mg, 0.30 mmol) was dissolved in methanol (10 mL) and the solution bubbled through with N₂ for 2 minutes. 10% Pd/C (46 mg) was added. The flask was fitted with a septum and a N₂-filled balloon, carefully evacuated and filled with N₂. The N₂-filled balloon was substituted with a H₂-filled balloon, the flask was then carefully evacuated and filled with H₂ twice and the reaction mixture vigorously stirred at rt for 4 h. The flask was the carefully evacuated and filled with N₂ twice, the reaction mixture filtered through Celite, concentrated and purified by flash chromatography (1%-5% methanol in DCM) to yield compound 1048. ¹H-NMR (DMSO-d₆) δ 11.84 (bs, 1H), 9.78 (bs, 1H), 7.54 (s, 1H), 7.34 (m, 1H), 7.2-7.07 (m, 2H), 7.05-6.95 (m, 3H), 6.79 (m, 2H).

### Example 49

### 6,7-difluoro-3-(4-hydroxyphenyl)-3-morpholinoindolin-2-one (compound 1049)

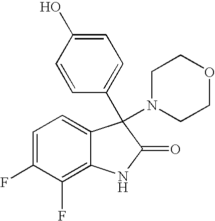

Compound 50 (82 mg, 0.23 mmol) was dissolved in DCM under nitrogen, cooled to −78° C. and BBr₃-solution (1.0 M, 341 μl, 0.35 mmol) was added dropwise with stirring. The reaction mixture was gradually allowed to reach room temperature and stirred on. The resulting precipitate was filtered off, washed with DCM and purified by chromatography (CHCl₃:MeOH:NH₃ (25%) 98:2:0.2) to afford compound 1049. ¹H-NMR (MeOD) δ 7.23 (m, 2H), 7.01 (m, 1H), 6.83 (m, 1H), 6.66 (m, 2H), 3.57 (m, 4H), 2.45 (m, 2H).

### Example 50

### 3-(4-hydroxyphenyl)-3-(thiazol-2-yl)-7-(trifluoromethyl)indolin-2-one (compound 1050)

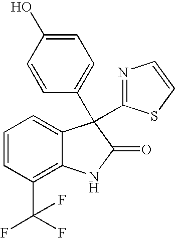

General procedure 2. Starting material: compound 51. ¹H-NMR (DMSO-d₆) δ 11.36 (bs, 1H), 9.64 (bs, 1H), 7.80 (d, 1H), 7.75 (d, 1H), 7.70 (d, 1H), 7.59 (d, 1H), 7.59 (d, 1H), 7.23 (t, 1H), 7.07 (m, 2H), 6.75 (m, 2H).

### Example 51

### 7-chloro-3-cyclohexyl-3-(4-hydroxyphenyl)-6-methylindolin-2-one (compound 1051)

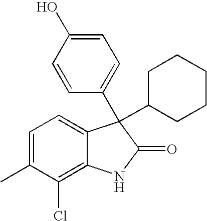

General procedure 2. Starting material: compound 52. ¹H-NMR (DMSO-d₆) δ 10.72 (s, 1H), 9.35 (bs, 1H), 7.18 (d, 1H), 7.12 (m, 2H), 7.02 (d, 1H), 6.70 (m, 2H), 2.34 (s, 3H), 2.28 (m, 1H), 1.60 (m, 3H), 1.41 (m, 2H), 1.3-0.8 (m, 4H), 0.70 (m, 1H).

### Example 52

### 7-chloro-3-cyclopentyl-3-(4-hydroxyphenyl)-6-methylindolin-2-one (compound 1052)

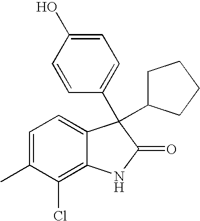

General procedure 2. Starting material: compound 53. ¹H-NMR (DMSO-d₆) δ 10.74 (s, 1H), 9.35 (s, 1H), 7.16 (d, 1H), 7.12 (m, 2H), 6.99 (d, 1H), 6.69 (m, 2H), 2.95 (m, 1H), 2.33 (s, 3H), 1.65-1.2 (m, 7H), 0.91 (m, 1H).

### Example 53

### 7-chloro-3-cycloheptyl-3-(4-hydroxyphenyl)-6-methylindolin-2-one (compound 1053)

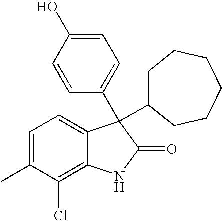

General procedure 2. Starting material: compound 54. ¹H-NMR (DMSO-d₆) δ 10.70 (s, 1H), 9.36 (s, 1H), 7.16 (d, 1H), 7.09 (m, 2H), 7.02 (d, 1H), 6.69 (m, 2H), 2.47 (m, 1H), 2.34 (s, 3H), 1.65-1.2 (m, 11H), 0.85 (m, 1H).

### Example 54

### 3-cyclohexyl-6-hydroxy-3-(4-hydroxyphenyl)-7-methylindolin-2-one (compound 1054)

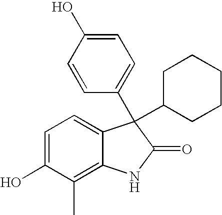

Compound 1046 was suspended in 37% aqueous HBr and heated to 120° C. in a microwave oven for 3 times 10 minutes, concentrated twice with toluene and purified by chromatography (1%-5% MeOH in DCM) to afford compound 1054. MS [M+H]⁺=338.1 [M−H]⁺=336.1

### Example 55

### 7-bromo-3-cyclopentyl-3-(4-hydroxyphenyl)-6-methylindolin-2-one (compound 1055)

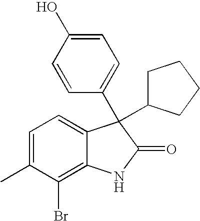

General procedure 2. Starting material: compound 55. ¹H-NMR (DMSO-d₆) δ 10.60 (s, 1H), 9.35 (s, 1H), 7.19 (d, 1H), 7.12 (m, 2H), 6.99 (m, 1H), 6.69 (m, 2H), 2.95 (m, 1H), 2.35 (s, 3H), 1.6-1.2 (m, 7H), 0.92 (m, 1H).

### Example 56

### 7-bromo-3-cyclohexyl-3-(4-hydroxyphenyl)-6-methylindolin-2-one (compound 1056)

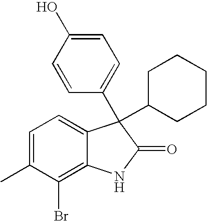

General procedure 2. Starting material: compound 56. ¹H-NMR (DMSO-d₆) δ 10.57 (s, 1H), 9.36 (s, 1H), 7.21 (d, 1H), 7.12 (m, 2H), 7.03 (m, 1H), 6.70 (m, 2H), 2.35 (s, 3H), 2.30 (m, 1H), 1.59 (m, 3H), 1.41 (m, 2H), 1.16 (m, 3H), 0.96 (m, 1H), 0.70 (m, 1H).

### Example 57

### 7-bromo-3-cycloheptyl-3-(4-hydroxyphenyl)-6-methylindolin-2-one (compound 1057)

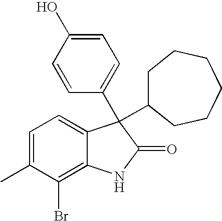

General procedure 2. Starting material: compound 57. ¹H-NMR (DMSO-d₆) δ 10.55 (s, 1H), 9.36 (s, 1H), 7.19 (d, 1H), 7.09 (m, 2H), 7.02 (m, 1H), 6.70 (m, 2H), 2.49 (m, 1H), 2.36 (s, 3H), 1.7-1.2 (m, 11H), 0.85 (m, 1H).

### Example 58

### 3-cyclopentyl-3-(4-hydroxyphenyl)-7-methylindolin-2-one (compound 1058)

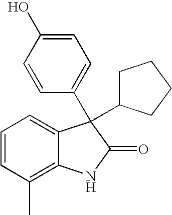

General procedure 2. Starting material: compound 58. ¹H-NMR (MeOD) δ 7.19 (m, 2H), 7.12 (m, 2H), 6.99 (t, 1H), 6.71 (m, 2H), 3.07 (m, 1H), 2.31 (s, 3H), 1.8-1.2 (m, 7H), 0.94 (m, 1H). MS [M+Na]⁺=330.1, [M−H]⁻=306.1

### Example 59

### 3-cyclohexyl-3-(4-hydroxyphenyl)-7-methylindolin-2-one (compound 1059)

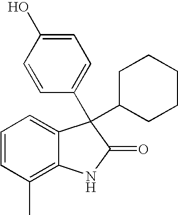

General procedure 2. Starting material: compound 59. ¹H-NMR (DMSO-d₆) δ 10.42 (bs, 1H), 9.32 (bs, 1H), 7.14 (m, 3H), 7.05 (d, 1H), 6.94 (t, 1H), 6.69 (m, 2H), 2.30 (m, 1H), 2.21 (s, 3H), 1.7-0.8 (m, 9H), 0.70 (m, 1H).

### Example 60

### 3-cycloheptyl-3-(4-hydroxyphenyl)-7-methylindolin-2-one (compound 1060)

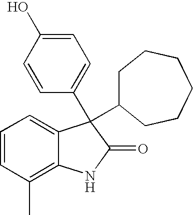

General procedure 2. Starting material: compound 60. ¹H-NMR (DMSO-d₆) δ 10.39 (bs, 1H), 9.31 (bs, 1H), 7.11 (m, 3H), 7.05 (d, 1H), 6.93 (t, 1H), 6.68 (m, 2H), 2.46 (m, 1H), 2.21 (s, 3H), 1.7-1.2 (m, 9H), 0.85 (m, 1H).

### Example 61

### 7-bromo-3-cyclopentyl-3-(4-hydroxyphenyl)indolin-2-one (compound 1061)

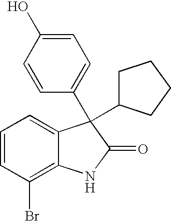

General procedure 2. Starting material: compound 61. ¹H-NMR (DMSO-d₆) δ 10.73 (s, 1H), 9.37 (s, 1H), 7.44 (d, 1H), 7.30 (d, 1H), 7.12 (m, 2H), 6.97 (t, 1H), 6.70 (m, 2H), 2.97 (m, 1H), 1.6-1.2 (m, 7H), 0.98 (m, 1H).

### Example 62

### 7-bromo-3-cyclohexyl-3-(4-hydroxyphenyl)indolin-2-one (compound 1062)

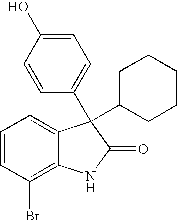

General procedure 2. Starting material: compound 62. ¹H-NMR (DMSO-d₆) δ 10.70 (s, 1H), 9.38 (s, 1H), 7.50 (d, 1H), 7.33 (d, 1H), 7.12 (m, 2H), 7.00 (t, 1H), 6.71 (m, 2H), 2.33 (m, 1H), 1.62 (m, 3H), 1.41 (m, 2H), 1.16 (m, 3H) 0.97 (m, 1H), 0.74 (m, 1H).

### Example 63

### 7-bromo-3-cycloheptyl-3-(4-hydroxyphenyl)indolin-2-one (compound 1063)

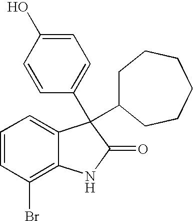

General procedure 2. Starting material: compound 63. ¹H-NMR (DMSO-d₆) δ 10.67 (bs, 1H), 9.38 (s, 1H), 7.45 (d, 1H), 7.30 (d, 1H), 7.09 (m, 2H), 6.99 (t, 1H), 6.70 (m, 2H), 2.53 (m, 1H), 1.7-1.2 (m, 11H), 0.86 (m, 1H).

### Example 64

### 3-cyclooctyl-3-(4-hydroxyphenyl)-7-(trifluoromethyl)indolin-2-one (compound 1064)

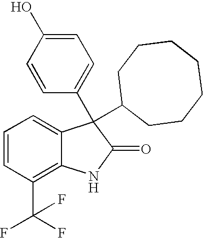

General procedure 2. Starting materials: compound 64. ¹H-NMR (DMSO-d₆) δ 10.89 (bs, 1H), 9.41 (s, 1H), 7.63 (d, 1H), 7.54 (d, 1H), 7.21 (t, 1H), 7.11 (m, 2H), 6.72 (m, 2H), 2.68 (m, 1H), 1.7-1.1 (m, 13H), 0.93 (m, 1H).

### Example 65

### 7-chloro-3-cyclooctyl-3-(4-hydroxyphenyl)-6-methylindolin-2-one (compound 1065)

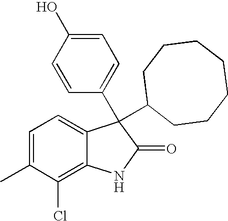

General procedure 2. Starting materials: compound 65. ¹H-NMR (DMSO-d₆) δ 10.70 (bs, 1H), 9.37 (s, 1H), 7.18 (d, 1H), 7.11 (m, 2H), 7.01 (d, 1H), 6.70 (m, 2H), 2.62 (m, 1H), 2.33 (s, 3H), 1.65-1.05 (m, 13H), 0.87 (m, 1H).

### Example 66

### 3-cyclopentyl-3-(4-hydroxyphenyl)-5,7-dimethylindolin-2-one (compound 1066)

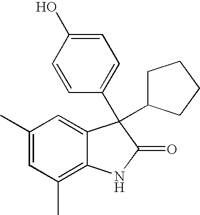

General procedure 2. Starting material: compound 66. ¹H-NMR (DMSO-d₆) δ 10.35 (bs, 1H), 9.29 (s, 1H), 7.13 (m, 2H), 6.90 (s, 1H), 6.84 (s, 1H), 6.68 (m, 2H), 2.92 (m, 1H), 2.24 (s, 3H), 2.19 (s, 3H), 1.6-1.2 (m, 7H), 0.98 (m, 1H).

### Example 67

### 3-cyclohexyl-3-(4-hydroxyphenyl)-5,7-dimethylindolin-2-one (compound 1067)

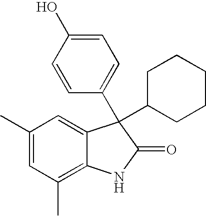

General procedure 2. Starting material: compound 67. ¹H-NMR (DMSO-d₆) δ 10.32 (bs, 1H), 9.29 (s, 1H), 7.14 (m, 2H), 6.93 (s, 1H), 6.85 (s, 1H), 6.68 (m, 2H), 2.30 (m, 1H), 2.27 (s, 3H), 2.17 (s, 3H), 1.60 (m, 3H), 1.41 (m, 2H), 1.15 (m, 3H), 0.97 (m, 1H), 0.76 (m, 1H).

### Example 68

### 3-cycloheptyl-3-(4-hydroxyphenyl)-5,7-dimethylindolin-2-one (compound 1068)

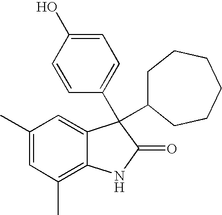

General procedure 2. Starting material: compound 68. ¹H-NMR (DMSO-d₆) δ 10.29 (bs, 1H), 9.32 (bs, 1H), 7.11 (m, 2H), 6.91 (s, 1H), 6.85 (s, 1H), 6.68 (m, 2H), 2.47 (m, 1H), 2.26 (s, 3H), 2.17 (s, 3H), 1.7-1.1 (m, 11H), 0.86 (m, 1H).

### Example 69

### 3-cyclopentyl-3-(4-hydroxyphenyl)-5-methoxy-7-methylindolin-2-one (compound 1069)

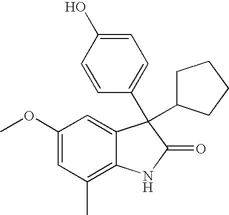

General procedure 2. Starting material: compound 69. ¹H-NMR (DMSO-d₆) δ 10.30 (bs, 1H), 9.30 (bs, 1H), 7.14 (m, 2H), 6.67 (m, 4H), 3.69 (s, 3H), 2.95 (m, 1H), 2.21 (s, 3H), 1.6-1.2 (m, 7H), 1.00 (m, 1H).

### Example 70

### 3-cyclohexyl-3-hydroxy-5-methoxy-7-methylindolin-2-one (compound 1070)

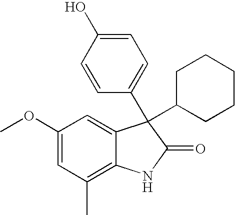

General procedure 2. Starting material: compound 70. ¹H-NMR (DMSO-d₆) δ 10.27 (bs, 1H), 9.30 (bs, 1H), 7.14 (m, 2H), 6.68 (m, 4H), 3.71 (s, 3H), 2.30 (m, 1H), 2.20 (s, 3H), 1.59 (m, 3H), 1.39 (m, 2H), 1.15 (m, 3H), 0.98 (m, 1H), 0.77 (m, 1H).

### Example 71

### 3-cycloheptyl-3-(4-hydroxyphenyl)-5-methoxy-7-methylindolin-2-one (compound 1071)

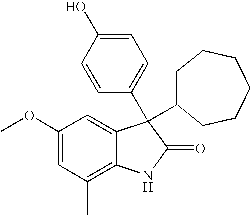

General procedure 2. Starting material: compound 71. ¹H-NMR (DMSO-d₆) δ 10.29 (bs, 1H), 9.31 (bs, 1H), 7.11 (m, 2H), 6.68 (m, 4H), 3.71 (s, 3H), 2.45 (m, 1H), 2.20 (s, 3H), 1.7-1.15 (m, 11H), 0.87 (m, 1H).

### Example 72

### 5-chloro-3-cyclopentyl-3-(4-hydroxyphenyl)-7-methylindolin-2-one (compound 1072)

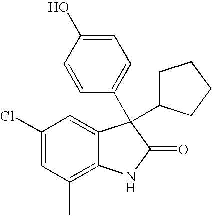

General procedure 2. Starting material: compound 72. ¹H-NMR (DMSO-d₆) δ 10.65 (bs, 1H), 9.35 (bs, 1H), 7.13 (m, 4H), 6.70 (m, 2H), 2.97 (m, 1H), 2.23 (s, 3H), 1.65-1.35 (m, 6H), 1.24 (m, 1H), 0.90 (m, 1H).

### Example 73

### 5-chloro-3-cyclohexyl-3-(4-hydroxyphenyl)-7-methylindolin-2-one (compound 1073)

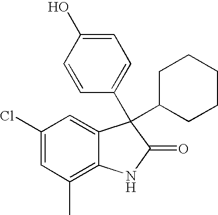

General procedure 2. Starting material: compound 73. ¹H-NMR (DMSO-d₆) δ 10.62 (bs, 1H), 9.36 (bs, 1H), 7.13 (m, 4H), 6.71 (m, 2H), 2.35 (m, 1H), 2.21 (s, 3H), 1.61 (m, 3H), 1.39 (m, 2H), 1.3-0.9 (m, 4H), 0.81 (m, 1H).

### Example 74

### 5-chloro-3-cycloheptyl-3-hydroxy-7-methylindolin-2-one (compound 1074)

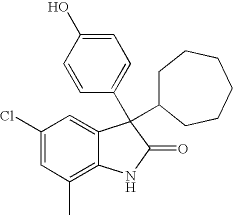

General procedure 2. Starting material: compound 74. ¹H-NMR (DMSO-d₆) δ 10.59 (bs, 1H), 9.38 (s, 1H), 7.11 (m, 4H), 6.71 (m, 2H), 2.49 (m, 1H), 2.21 (s, 3H), 1.7-1.15 (m, 11H), 0.89 (m, 1H).

### Example 75

### 3-cyclopentyl-5-fluoro-3-(4-hydroxyphenyl)-7-methylindolin-2-one (compound 1075)

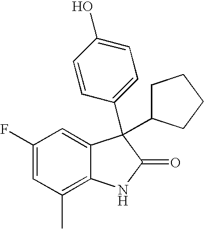

General procedure 2. Starting material: compound 75. ¹H-NMR (DMSO-d₆) δ 10.52 (bs, 1H), 9.33 (bs, 1H), 7.12 (m, 2H), 6.95 (dd, 1H), 6.89 (dd, 1H), 6.70 (m, 2H), 2.95 (m, 1H), 2.22 (s, 3H), 1.6-0.95 (m, 7H), 0.87 (m, 1H).

### Example 76

### 3-cyclohexyl-5-fluoro-3-(4-hydroxyphenyl)-7-methylindolin-2-one (compound 1076)

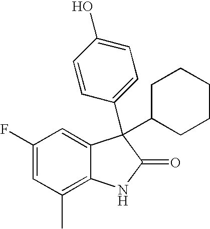

General procedure 2. Starting material: compound 76. ¹H-NMR (DMSO-d₆) δ 10.49 (bs, 1H), 9.34 (bs, 1H), 7.14 (m, 2H), 7.01 (dd, 1H), 6.93 (dd, 1H), 6.70 (m, 2H), 2.32 (m, 1H), 2.22 (s, 3H), 1.60 (m, 3H), 1.40 (m, 2H), 1.3-0.9 (m, 4H), 0.78 (m, 1H).

### Example 77

### 3-cycloheptyl-5-fluoro-3-(4-hydroxyphenyl)-7-methylindolin-2-one (compound 1077)

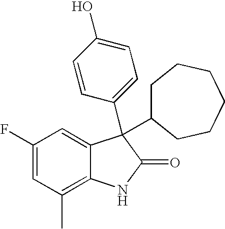

General procedure 2. Starting material: compound 77. ¹H-NMR (DMSO-d₆) δ 10.46 (bs, 1H), 9.35 (s, 1H), 7.11 (m, 2H), 6.95 (m, 2H), 6.70 (m, 2H), 2.49 (m, 1H), 2.22 (s, 3H), 1.7-1.1 (m, 11H), 0.87 (m, 1H).

### Example 78

### 3-cyclopentyl-6-fluoro-3-(4-hydroxyphenyl)-5,7-dimethylindolin-2-one (compound 1078)

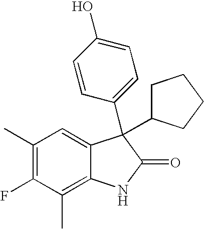

General procedure 2. Starting material: compound 78. ¹H-NMR (DMSO-d₆) δ 10.55 (bs, 1H), 9.32 (bs, 1H), 7.12 (m, 2H), 7.00 (d, 1H), 6.68 (m, 2H), 2.92 (m, 1H), 2.18 (d, 3H), 2.15 (d, 3H), 1.6-1.2 (m, 7H), 0.95 (m, 1H).

### Example 79

### 3-cyclohexyl-6-fluoro-3-(4-hydroxyphenyl)-5,7-dimethylindolin-2-one (compound 1079)

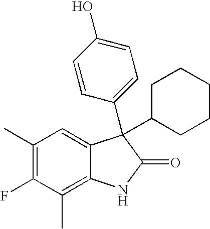

General procedure 2. Starting material: compound 79. ¹H-NMR (DMSO-d₆) δ 10.53 (bs, 1H), 9.32 (bs, 1H), 7.12 (m, 2H), 7.03 (d, 1H), 6.69 (m, 2H), 2.28 (m, 1H), 2.22 (d, 3H), 2.13 (d, 3H), 1.60 (m, 3H), 1.39 (m, 2H), 1.16 (m, 3H), 0.98 (m, 1H), 0.74 (m, 1H).

### Example 80

### 3-cycloheptyl-6-fluoro-3-(4-hydroxyphenyl)-5,7-dimethylindolin-2-one (compound 1080)

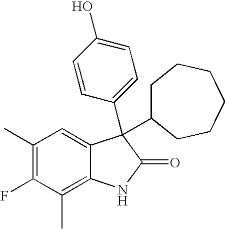

General procedure 2. Starting material: compound 80. ¹H-NMR (DMSO-d₆) δ 10.50 (bs, 1H), 9.32 (s, 1H), 7.09 (m, 2H), 7.00 (d, 1H), 6.69 (m, 2H), 2.46 (m, 1H), 2.20 (d, 3H), 2.13 (d, 3H), 1.75-1.15 (m, 11H), 0.85 (m, 1H).

### Example 81

### 3-cyclopentyl-3-(4-hydroxyphenyl)-7-methyl-6-(trifluoromethyl)indolin-2-one (compound 1081)

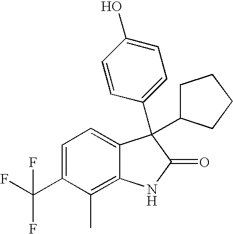

General procedure 2. Starting material: compound 81. ¹H-NMR (MeOD) δ 7.28 (d, 1H), 7.17 (d, 1H), 7.07 (m, 2H), 2.98 (m, 1H), 2.29 (d, 3H), 1.7-1.1 (m, 7H), 0.90 (m, 1H).

### Example 82

### 3-cyclohexyl-3-(4-hydroxyphenyl)-7-methyl-6-(trifluoromethyl)indolin-2-one (compound 1082)

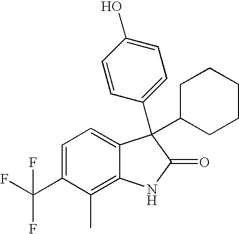

General procedure 2. Starting material: compound 82. ¹H-NMR (DMSO-d₆) δ 10.79 (bs, 1H), 9.39 (bs, 1H), 7.37 (m, 2H), 7.13 (m, 2H), 6.71 (m, 2H), 2.37 (m, 1H), 2.32 (d, 3H), 1.58 (m, 3H), 1.42 (m, 2H), 1.2 (m, 3H), 1.01 (m, 1H), 0.75 (m, 1H).

### Example 83

### 3-cycloheptyl-3-(4-hydroxyphenyl)-7-methyl-6-(trifluoromethyl)indolin-2-one (compound 1083)

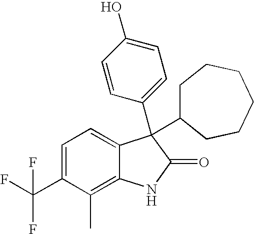

General procedure 2. Starting material: compound 83. ¹H-NMR (MeOD) δ 7.42 (d, 1H), 7.31 (d, 1H), 7.15 (m, 2H), 6.74 (m, 2H), 2.65 (m, 1H), 2.38 (d, 3H), 1.8-1.2 (m, 11H), 0.88 (m, 1H).

### Example 84

### 3-cyclopentyl-3-(4-hydroxyphenyl)-5-methoxy-6,7-dimethylindolin-2-one (compound 1084)

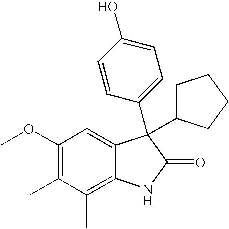

General procedure 2. Starting material: compound 84. ¹H-NMR (DMSO-d₆) δ 10.22 (bs, 1H), 9.28 (bs, 1H), 7.13 (m, 2H), 6.69 (m, 3H), 3.69 (s, 3H), 2.96 (m, 1H), 2.15 (s, 3H), 2.08 (s, 3H), 1.65-1.1 (m, 7H), 0.96 (m, 1H).

### Example 85

### 3-cyclohexyl-3-(4-hydroxyphenyl)-5-methoxy-6,7-dimethylindolin-2-one (compound 1085)

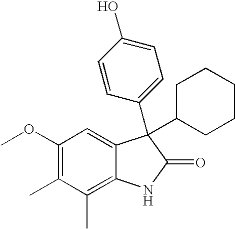

General procedure 2. Starting material: compound 85. ¹H-NMR (DMSO-d₆) δ 10.19 (bs, 1H), 9.29 (bs, 1H), 7.14 (m, 2H), 6.70 (m, 3), 3.32 (s, 3H), 2.31 (m, 1H), 2.13 (s, 3H), 2.09 (s, 3H), 1.7-1.1 (m, 8H), 0.98 (m, 1H), 0.76 (m, 1H).

### Example 86

### 3-cycloheptyl-3-(4-hydroxyphenyl)-5-methoxy-6,7-dimethylindolin-2-one (compound 1086)

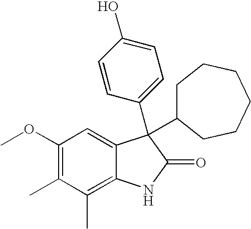

General procedure 2. Starting material: compound 86. ¹H-NMR (DMSO-d₆) δ 10.16 (bs, 1H), 9.28 (bs, 1H), 7.11 (m, 2H), 6.69 (m, 3H), 3.72 (s, 3H), 2.46 (m, 1H), 2.13 (s, 3H), 2.09 (s, 3H), 1.7-1.1 (m, 11H), 0.88 (m, 1H).

### Example 87

### 3-cycloheptyl-3-(4-hydroxyphenyl)-6-methoxy-7-methylindolin-2-one (compound 1087)

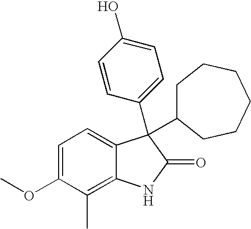

General procedure 2. Starting material: compound 45. ¹H-NMR (DMSO-d₆) δ 10.35 (bs, 1H), 9.29 (bs, 1H), 7.11 (m, 2H), 7.05 (d, 1H), 6.67 (m, 2H), 6.61 (d, 1H), 3.79 (s, 3H), 2.45 (m, 1H), 2.05 (s, 3H), 1.7-1.2 (m, 11H), 0.83 (m, 1H).

### Example 88

### In vitro Cell Proliferation Assay (WST Assay)

MCF-7 cells were seeded in 96-well plates at 3×10³ cells/well in 100 μL of culture medium, 8 wells were left empty for media only controls.

After 24 h the compound titrations were performed, in a separate dilution plate, by serially diluting the compounds of general formula (I) in culture medium. A 100 μL of each dilution was added to the plated cells, this was done in triplicate, and controls (e.g. DMSO and blanks) were included. The plates were incubated for 24 h at 37° C. in a CO₂ incubator. The compound titrations were repeated in a separate dilution plate after 24 h. The media plus compound from the assay plates were then aspirated. A 100 μL of media was then added to all wells, followed by 100 μL of each compound dilution. The plates were incubated for a further 48 h at 37° C. in a CO₂ incubator (total incubation time 72 h). The number of viable cells was then assessed using Cell Proliferation Reagent WST-1. 10 μL of WST-1 reagent added to each well and incubated for one to four hours at 37° C. in CO₂ incubator. The absorbance was measured (450 nm/690 nm).

The activity of compounds of general formula (I) in reducing the number of viable cells was calculated as:

% activity=[(S^{c}−B)/(S^{o}−B)]×100

S^{c} denotes signal measured in the presence of test compound, S^{o} denotes signal detected in the absence of compound, and B denotes background signal, measured in blank wells containing medium only. Analyse data using GraphPad Prism.

Results can be seen in Table 1.

TABLE 1
In vitro cell proliferation assay (WST-assay as described in Example 88)

| | IC₅₀ (nM) |
|---|---|
| Compound No. | for MCF-7 |
| Reference compound (Compound 41 in WO | 8.4 |
| 2005/097107) 6,7-Difluoro-3,3-bis-(4- | |
| hydroxy-phenyl)-1,3-dihydro-indol-2-one | |
| Compound 1006 | 34.5 |
| Compound 1010 | 7.3 |
| Compound 1018 | 3.4 |
| Compound 1021 | 17.5 |
| Compound 1022 | 5.2 |
| Compound 1023 | 4.0 |
| Compound 1025 | 25.9 |
| Compound 1027 | 3.9 |
| Compound 1028 | >500 |
| Compound 1033 | 10.2 |
| Compound 1034 | 9.1 |
| Compound 1039 | 5.7 |
| Compound 1043 | 19.1 |
| Compound 1046 | 12.6 |
| Compound 1053 | 3.5 |
| Compound 1056 | 10.5 |
| Compound 1059 | 32.9 |
| Compound 1062 | 6.8 |
| Compound 1070 | 10.0 |
| Compound 1087 | 4.7 |

1-31. (canceled)

32. A compound of the general formula (I) 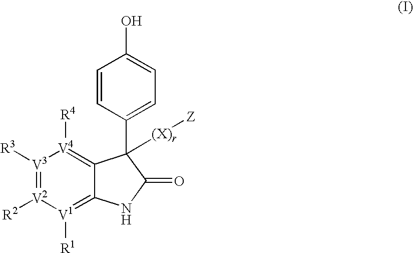
wherein
r is 0 or 1;
X is selected from —CH₂—, —O—, —S—, —S(O)—, —S(O)₂— and —NR⁵—, wherein R⁵ is selected from hydrogen and optionally substituted C₁₋₆-alkyl;
Z is selected from optionally substituted C₁₋₁₂-alkyl, optionally substituted C₃₋₁₂-cycloalkyl, optionally substituted C₂₋₁₂-alkenyl, optionally substituted C₃₋₁₂-cycloalkenyl, optionally substituted C₂₋₁₂-alkynyl, optionally substituted heterocyclyl, optionally substituted aryl and optionally substituted heteroaryl;
with the proviso that Z is not para-mono-substituted phenyl when r is 0;
V¹, V², V³, and V⁴ independently are selected from a carbon atom, a non-quaternary nitrogen atom, an oxygen atom, and a sulphur atom, and where V⁴ further may be selected from a bond, so that -V¹-V²-V³-V⁴- together with the atoms to which V¹ and V⁴ are attached form an aromatic or heteroaromatic ring;
R¹, R², R³, and R⁴, when attached to a carbon atom, independently are selected from hydrogen, optionally substituted C₁₋₁₂-alkyl, optionally substituted C₃₋₁₂-cycloalkyl, optionally substituted C₂₋₁₂-alkenyl, optionally substituted C₃₋₁₂-cycloalkenyl, hydroxy, optionally substituted C₁₋₁₂-alkoxy, optionally substituted C₂₋₁₂-alkenyloxy, carboxy, optionally substituted C₁₋₁₂-alkoxycarbonyl, optionally substituted C₁₋₁₂-alkylcarbonyl, optionally substituted C₁₋₁₂-alkylcarbonyloxy, formyl, amino, mono- and di(C₁₋₁₂-alkyl)amino, carbamoyl, mono- and di(C₁₋₁₂-alkyl)aminocarbonyl, alkylcarbonylamino, C₁₋₁₂-alkylsulphonylamino, cyano, carbamido, mono- and di(C₁₋₁₂-alkyl)-aminocarbonylamino, C₁₋₁₂-alkanoyloxy, C₁₋₁₂-alkylsulphonyl, C₁₋₁₂-alkylsulphinyl, aminosulphonyl, mono- and di(C₁₋₁₂-alkyl)aminosulphonyl, nitro, optionally substituted C₁₋₁₂-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₁₂-alkyl as an amino substituent is optionally substituted with hydroxy, C₁₋₁₂-alkoxy, amino, mono- and di(C₁₋₁₂-alkyl)amino, carboxy, C₁₋₁₂-alkylcarbonylamino, C₁₋₁₂-alkylamino-carbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₁₂-alkyl, hydroxy, oxide, optionally substituted C₁₋₁₂-alkoxy, optionally substituted C₁₋₁₂-alkoxycarbonyl, optionally substituted C₁₋₁₂-alkylcarbonyl, formyl, mono- and di(C₁₋₁₂-alkyl)aminocarbonyl, amino, C₁₋₁₂-alkylcarbonylamino, mono- and di(C₁₋₁₂-alkyl)amino, C₁₋₁₂-alkylsulphonyl, C₁₋₁₂-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₁₂-alkyl as an amino substituent is optionally substituted with hydroxy, C₁₋₁₂-alkoxy, amino, mono- and di(C₁₋₁₂-alkyl)amino, carboxy, C₁₋₁₂-alkylcarbonylamino, C₁₋₁₂-alkylaminocarbonyl, or halogen(s), and wherein any aryl, heterocyclyl and heteroaryl may be optionally substituted;
or R¹ and R² together with the carbon atoms to which they are attached form a ring;
with the proviso that at least one of the substituents R¹, R², R³, and R⁴ is not hydrogen;
and pharmaceutically acceptable salts and prodrugs thereof.

33. The compound according to claim 32, wherein Z is selected from optionally substituted C₁₋₁₂-alkyl, optionally substituted C₃₋₁₂-cycloalkyl, optionally substituted C₂₋₁₂-alkenyl, optionally substituted C₃₋₁₂-cycloalkenyl, optionally substituted C₂₋₁₂-alkynyl, and optionally substituted heterocyclyl.

34. The compound according to claim 32, wherein Z is optionally substituted heteroaryl.

35. The compound according to claim 32, wherein Z is aryl.

36. The compound according to claim 32, wherein Z is di- or tri-substituted aryl.

37. The compound according to claim 32, wherein r is 1 and X is —CH₂—.

38. The compound according to claim 32, wherein r is 0.

39. The compound according to claim 32, wherein each of V¹, V², V³, and V⁴ represents a carbon atom (a benzene ring), or V³ represents a nitrogen atom and each of V¹, V², and V⁴ represents a carbon atom (a pyridine ring).

40. The compound according to claim 39, wherein each of V¹, V², V³, and V⁴ represents a carbon atom.

41. The compound according to claim 32, wherein R¹ is selected from halogen, C₁₋₆-alkyl, trifluoromethyl and C₁₋₆-alkoxy, when V¹ is a carbon atom.

42. The compound according to claim 32, wherein R² is selected from halogen, optionally substituted C₁₋₆-alkyl, and optionally substituted C₁₋₆-alkoxy, when V² is a carbon atom.

43. The compound according to claim 32, wherein R³ is selected from hydrogen, optionally substituted C₁₋₆-alkoxy, halogen, cyano, optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulphonyl, when V³ is a carbon atom.

44. The compound according to claim 32, wherein R⁴ is hydrogen, when V⁴ is a carbon atom.

45. The compound according to claim 32, wherein at least two of the substituents R¹, R², R³, and R⁴ are not hydrogen.

46. The compounds according to claim 32, wherein R¹ and R² together with the carbon atoms to which they are attached form a ring selected from aromatic rings, carbocyclic rings, heterocyclic rings and heteroaromatic rings, in particular aromatic rings, heterocyclic rings and heteroaromatic rings.

47. The compound according to claim 32 having the general formula (Ia) 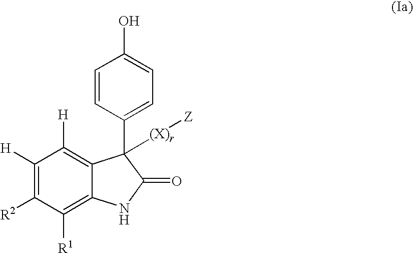
wherein Z, R¹ and R² are as defined in claim 32, with the proviso that none of R¹ and R² are hydrogen.

48. A compound selected from the group consisting of:
3-ethynyl-6,7-difluoro-3-(4-hydroxyphenyl)indolin-2-one
3-benzyl-6,7-difluoro-3-(4-hydroxyphenyl)indoline-2-one
6,7-difluoro-3-(4-hydroxyphenyl)-3-methylindolin-2-one
3-cyclopentyl-6,7-difluoro-3-(4-hydroxyphenyl)indolin-2-one
3-(cyclohexylmethyl)-6,7-difluoro-3-(4-hydroxyphenyl)indolin-2-one
6,7-difluoro-3-(4-hydroxyphenyl)-3-(pyridin-4-yl)indolin-2-one
6,7-difluoro-3-(4-hydroxyphenyl)-3-isopropylindolin-2-one
6,7-difluoro-3-(4-hydroxyphenyl)-3-(thiophen-2-yl)indolin-2-one
3-butyl-6,7-difluoro-3-(4-hydroxyphenyl)indolin-2-one
3-cyclohexyl-6,7-difluoro-3-(4-hydroxyphenyl)indolin-2-one
6,7-difluoro-3-(4-hydroxyphenyl)-3-propylindolin-2-one
6,7-difluoro-3-(4-hydroxyphenyl)-3-pentylindolin-2-one
6,7-difluoro-3-(4-hydroxyphenyl)-3-pentylindolin-2-one
6,7-difluoro-3-(4-hydroxyphenyl)-3-(pyridin-3-yl)indolin-2-one
6,7-difluoro-3-(4-hydroxyphenyl)-3-(pyridin-4-yl N-oxide)indolin-2-one
3-(but-3-en-2-yl)-6,7-difluoro-3-(4-hydroxyphenyl)indolin-2-one
3-sec-butyl-6,7-difluoro-3-(4-hydroxyphenyl)indolin-2-one
3-cycloheptyl-6,7-difluoro-3-(4-hydroxyphenyl)indolin-2-one
3-(1-(benzyloxy)-1H-pyrazol-4-yl)-6,7-difluoro-3-(4-hydroxyphenyl)indolin-2-one
6,7-difluoro-3-(1-hydroxy-1H-pyrazol-4-yl)-3-(4-hydroxyphenyl)indolin-2-one
3-cyclohexyl-3-(4-hydroxyphenyl)-7-(trifluoromethyl)indolin-2-one
3-(3,4-difluorophenyl)-6,7-difluoro-3-(4-hydroxyphenyl)indolin-2-one
6,7-difluoro-3-(3-fluor-4-methylphenyl)-3-(4-hydroxyphenyl)indolin-2-one
6-chloro-3-cyclohexyl-3-(4-hydroxyphenyl)-7-methylindolin-2-one
3-cyclohexyl-3-(4-hydroxyphenyl)-6,7-dimethylindolin-2-one
3-(cyclopentylmethyl)-6,7-difluoro-3-(4-hydroxyphenyl)indolin-2-one
3-cyclohexyl-6,7-difluoro-3-(4-hydroxyphenyl)indolin-2-one (R-enantiomer)
3-cyclohexyl-6,7-difluoro-3-(4-hydroxyphenyl)indolin-2-one (S-enantiomer)
6,7-difluoro-3-(4-hydroxy-3-methylphenyl)-3-(4-hydroxyphenyl)indolin-2-one
6,7-difluoro-3-(4-hydroxy-2-methylphenyl)-3-(4-hydroxyphenyl)indolin-2-one
3-cyclooctyl-6,7-difluoro-3-(4-hydroxyphenyl)indolin-2-one
6,7-difluoro-3-(4-hydroxyphenyl)-3-(naphthalene-1-yl)indolin-2-one
6,7-difluoro-3-(4-hydroxyphenyl)-3-(naphthalene-2-yl)indolin-2-one
3-cyclohexyl-7-fluoro-3-(4-hydroxyphenyl)-6-methylindolin-2-one
3-tert-butyl-6,7-difluoro-3-(4-hydroxyphenyl)indolin-2-one
6,7-difluoro-3-(4-hydroxyphenyl)-3-tert-pentylindolin-2-one
3-cyclopentyl-6-fluoro-3-(4-hydroxyphenyl)-7-methylindolin-2-one
3-cyclohexyl-6-fluoro-3-(4-hydroxyphenyl)-7-methylindolin-2-one
6-fluoro-3-(4-hydroxyphenyl)-7-methyl-3-pentylindolin-2-one
3-cycloheptyl-6-fluoro-3-(4-hydroxyphenyl)-7-methylindolin-2-one
3-cycloheptyl-3-(4-hydroxyphenyl)-7-(trifluoromethyl)indolin-2-one
3-cycloheptyl-3-(4-hydroxyphenyl)-6,7-dimethylindolin-2-one
6-chloro-3-cycloheptyl-3-(4-hydroxyphenyl)-7-methylindolin-2-one
3-cyclopentyl-3-(4-hydroxyphenyl)-6-methoxy-7-methylindolin-2-one
3-cyclohexyl-3-(4-hydroxyphenyl)-6-methoxy-7-methylindolin-2-one
3-(4-hydroxyphenyl)-3-(1H-imidazol-1-yl)-7-(trifluoromethyl)indolin-2-one
6,7-difluoro-3-(4-hydroxyphenyl)-3-(1H-imidazol-1-yl)indolin-2-one
6,7-difluoro-3-(4-hydroxyphenyl)-3-morpholinoindolin-2-one
3-(4-hydroxyphenyl)-3-(thiazol-2-yl)-7-(trifluoromethyl)indolin-2-one
7-chloro-3-cyclohexyl-3-(4-hydroxyphenyl)-6-methylindolin-2-one
7-chloro-3-cyclopentyl-3-(4-hydroxyphenyl)-6-methylindolin-2-one
7-chloro-3-cycloheptyl-3-(4-hydroxyphenyl)-6-methylindolin-2-one)
3-cyclohexyl-6-hydroxy-3-(4-hydroxyphenyl)-7-methylindolin-2-one
7-bromo-3-cyclopentyl-3-(4-hydroxyphenyl)-6-methyl indolin-2-one
7-bromo-3-cyclohexyl-3-(4-hydroxyphenyl)-6-methylindolin-2-one
7-bromo-3-cycloheptyl-3-(4-hydroxyphenyl)-6-methylindolin-2-one
3-cyclopentyl-3-(4-hydroxyphenyl)-7-methylindolin-2-one
3-cyclohexyl-3-(4-hydroxyphenyl)-7-methylindolin-2-one
3-cycloheptyl-3-(4-hydroxyphenyl)-7-methylindolin-2-one
7-bromo-3-cyclopentyl-3-(4-hydroxyphenyl)indolin-2-one
7-bromo-3-cyclohexyl-3-(4-hydroxyphenyl)indolin-2-one
7-bromo-3-cycloheptyl-3-(4-hydroxyphenyl)indolin-2-one
3-cyclooctyl-3-(4-hydroxyphenyl)-7-(trifluoromethyl)indolin-2-one
7-chloro-3-cyclooctyl-3-(4-hydroxyphenyl)-6-methylindolin-2-one
3-cyclopentyl-3-(4-hydroxyphenyl)-5,7-dimethylindolin-2-one
3-cyclohexyl-3-(4-hydroxyphenyl)-5,7-dimethylindolin-2-one
3-cycloheptyl-3-(4-hydroxyphenyl)-5,7-dimethylindolin-2-one
3-cyclopentyl-3-(4-hydroxyphenyl)-5-methoxy-7-methylindolin-2-one
3-cyclohexyl-3-hydroxy-5-methoxy-7-methylindolin-2-one
3-cycloheptyl-3-(4-hydroxyphenyl)-5-methoxy-7-methylindolin-2-one
5-chloro-3-cyclopentyl-3-(4-hydroxyphenyl)-7-methylindolin-2-one
5-chloro-3-cyclohexyl-3-(4-hydroxyphenyl)-7-methylindolin-2-one
5-chloro-3-cycloheptyl-3-hydroxy-7-methylindolin-2-one
3-cyclopentyl-5-fluoro-3-(4-hydroxyphenyl)-7-methylindolin-2-one
3-cyclohexyl-5-fluoro-3-(4-hydroxyphenyl)-7-methylindolin-2-one
3-cycloheptyl-5-fluoro-3-(4-hydroxyphenyl)-7-methylindolin-2-one
3-cyclopentyl-6-fluoro-3-(4-hydroxyphenyl)-5,7-dimethylindolin-2-one
3-cyclohexyl-6-fluoro-3-(4-hydroxyphenyl)-5,7-dimethylindolin-2-one
3-cycloheptyl-6-fluoro-3-(4-hydroxyphenyl)-5,7-dimethylindolin-2-one
3-cyclopentyl-3-(4-hydroxyphenyl)-7-methyl-6-(trifluoromethyl)indolin-2-one
3-cyclohexyl-3-(4-hydroxyphenyl)-7-methyl-6-(trifluoromethyl)indolin-2-one
3-cycloheptyl-3-(4-hydroxyphenyl)-7-methyl-6-(trifluoromethyl)indolin-2-one
3-cyclopentyl-3-(4-hydroxyphenyl)-5-methoxy-6,7-dimethylindolin-2-one
3-cyclohexyl-3-(4-hydroxyphenyl)-5-methoxy-6,7-dimethylindolin-2-one
3-cycloheptyl-3-(4-hydroxyphenyl)-5-methoxy-6,7-dimethylindolin-2-one.

49. A pharmaceutical composition comprising a compound of the general formula (I) or (Ia) as defined in claim 32 and a pharmaceutically acceptable carrier.

50. A method of treating a mammal suffering from or being susceptible to cancer, the method comprising administering to the mammal a therapeutically effective amount of a compound of the general formula (I) or (Ia) as defined in claim 32.
